SEQUENCE LISTING (9 sequences)

SEQ ID NO: 1 - DNA, 19 bp, Artificial Sequence
  Primer
ccttctgtcttacttcata

SEQ ID NO: 2 - DNA, 21 bp, Artificial Sequence
  Primer
cagcaaagtgtgtgtgtggtt

SEQ ID NO: 3 - DNA, 21 bp, Artificial Sequence
  Primer
ctctgcactgtgttcagggac

SEQ ID NO: 4 - DNA, 21 bp, Artificial Sequence
  Primer
actgatgcagcccaggtactg

SEQ ID NO: 5 - DNA, 22 bp, Artificial Sequence
  Primer
ggaaggaccttcataggcttct

SEQ ID NO: 6 - DNA, 22 bp, Artificial Sequence
  Primer
tgcctatcttatccagctaggc

SEQ ID NO: 7 - DNA, 49 bp, Artificial Sequence
  Synthetically generated oligonucleotide
  misc_feature 22: n = 8-oxoguanine
tagacattgccattctcgatangatccggtcaaacctagacgaattccg

SEQ ID NO: 8 - DNA, 24 bp, Artificial Sequence
  Synthetically generated oligonucleotide
  misc_feature 14: n = 8-oxo guanine or uridine
ggcggcatgacccngaggcccatc

SEQ ID NO: 9 - DNA, 40 bp, Artificial Sequence
  Synthetically generated oligonucleotide
  misc_feature 22: n = 5-hydroxycytosine
aattgcgatctagctcgccagnagcgaccttatctgatga

(19) United States
(12) Patent Application Publication
(10) Pub. No.: US 2007/0219155 A1
(43) Pub. Date: Sep. 20, 2007

(54) TREATING TRINUCLEOTIDE REPEAT CONDITIONS

(75) Inventor: Cynthia T. McMurray, Oronoco, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 11/672,837

(22) Filed: Feb. 8, 2007

Related U.S. Application Data
(60) Provisional application No. 60/771,761, filed on Feb. 9, 2006.

(51) Int. Cl. A61K 48/00; C12Q 1/68; A01K 67/027; C12N 5/06; A61K 31/40

(52) U.S. Cl. 514/44; 514/408; 435/6; 435/354; 800/18

(57) ABSTRACT

This document provides methods and materials related to treating trinucleotide repeat conditions. For example, methods and materials for treating a trinucleotide repeat condition as well as methods and materials for identifying OGG1 polypeptide inhibitors that can be used to treat a trinucleotide repeat condition are provided.

### CLAIM OF PRIORITY

This application claims priority under 35 U.S.C. §119(e) to U.S. Provisional Patent Application Ser. No. 60/771,761, filed on Feb. 9, 2007, the entire contents of which are hereby incorporated by reference.

### STATEMENT AS TO FEDERALLY SPONSORED RESEARCH

Funding for the work described herein was provided by the federal government (National Institutes of Health grants NS40738 and GM066359), which may have certain rights in the invention.

### BACKGROUND

1. Technical Field

This document relates to methods and materials involved in treating trinucleotide repeat conditions such as Huntington's disease.

2. Background Information

Huntington's disease (HD) is an autosomal dominant condition that causes untold suffering for thousands of families. In the United States alone, about 30,000 people have HD, and estimates of HD prevalence are about one in every 10,000 people. At least 150,000 others have a 50 percent risk of developing the disease. HD results from degeneration of neurons in certain areas of the brain, which causes uncontrolled movements, loss of intellectual faculties, and emotional disturbance.

### SUMMARY

This document provides methods and materials related to treating trinucleotide repeat conditions. Typically, a trinucleotide repeat condition results from the expansion of a trinucleotide repeat, such as a CAG repeat. The methods and materials provided herein can be used to treat a trinucleotide repeat condition by, for example, reducing CAG repeat expansion. Reducing the expansion of a trinucleotide repeat can reduce the progression of the trinucleotide repeat condition, thereby allowing patients to maintain a quality of life.

This document is based, in part, on the discovery that an oxo-guanine glycosylase-1 (OGG1) polypeptide can be required for trinucleotide repeat expansion. This document also is based, in part, on the discovery that an inhibitor of an OGG1 polypeptide can be used to reduce trinucleotide repeat expansion.

In general, one aspect of this document features a method for reducing progression of a CAG repeat condition in a mammal. The method comprises, or consist essentially of, administering an inhibitor of an OGG1 polypeptide activity to the mammal under conditions wherein progression of symptoms of the CAG repeat condition is reduced as compared to progression of symptoms in a control mammal having the CAG repeat condition and not having been administered the inhibitor. The CAG repeat condition can be Huntington's disease. The mammal can be a human. The inhibitor can be a substituted pyrrolidine. The inhibitor can reduce the level of an OGG1 polypeptide in the mammal. The inhibitor can reduce the level of mRNA that encodes an OGG1 polypeptide in the mammal. The inhibitor can induce RNA interference. The inhibitor can be a nucleic acid comprising an 8-oxo-guanine base. Progression of the CAG repeat condition can be stopped in the mammal. The method can include diagnosing the mammal as having the CAG repeat condition.

In another embodiment, this document features a method for identifying a potential agent for reducing progression of a CAG repeat condition. The method comprises, or consists essentially of, (a) determining whether or not a test agent inhibits an OGG1 polypeptide activity, and (b) determining whether or not the test agent reduces CAG repeat expansion in a cell compared to a control cell not treated with the test agent, wherein the presence of a reduced CAG repeat expansion indicates that the test agent is the potential agent. The CAG repeat condition can be Huntington's disease. The test agent can be a substituted pyrrolidine. The test agent can comprise, or consist essentially of, nucleic acid. The step (a) can comprise, or consist essentially of, determining whether or not the test agent reduces the level of an OGG1 polypeptide in cells. The step (a) can comprise, or consist essentially of, determining whether or not the test agent reduces the level of mRNA that encodes an OGG1 polypeptide in cells. The test agent can be a nucleic acid comprising an 8-oxo-guanine base. The cell can be in vitro. The cell can be isolated from a mouse having the CAG repeat condition. The cell can be a mouse embryonic fibroblast cell. The cell can be in a mammal (e.g., a mouse). The mouse can be a mhtt mouse. The mhtt mouse can be an R6/1 mouse.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A, left panels contain GeneScan traces of the distributions of CAG repeats in tissues collected from hHD transgenic mice at the indicated ages (in weeks). The size of the repeats is determined by aligning the distributions with size standards. The vertical line designates the midpoint of the CAG repeat distribution in the youngest animals tested (3 weeks of age). Age-dependent expansion is defined as an increase in the major peak relative to that of the youngest age tested, which was 117 CAG repeats in this case. FIG. 1A, right panels contain graphs plotting the level of accumulated 8-oxoG lesions in tail, brain, and liver DNA from control (C) and hHD transgenic (Tg) mice at seven weeks of age (black bars) and 52 weeks of age (grey bars). Data collected from three to five mice are presented as the mean number of lesions with the standard deviation. FIG. 1B contains a series of graphs plotting the level of accumulated 5-hydroxycytosine (5-OHC), 3-methyladenene (3-MeA), formamidopyrimidine (FAPY) and Uracil lesions in brain and liver DNA from control (C) and hHD transgenic (Tg) mice at seven weeks of age (black bars) and 52 weeks of age (grey bars). FIG. 1C, top contains a schematic representation of the template used in the assay for 8-oxoG repair activity in tissue extracts from hHD animals and littermate controls. FIG. 1C, second panel from the top contains an autoradiograph of a polyacrylamide gel separating reaction products obtained after incubating the template with nuclear extracts from liver and brain of wild-type (Wt) and hHD (Tg) mice. FIG. 1C, middle contains a graph plotting the percent cut (an indicator of the percentage of 8-oxoG repair) of an 8-oxoG template following incubation with brain extracts from aging hHD mice (black circles) and their littermate controls (white circles). FIG. 1C, bottom panels contain graphs plotting the percentage of 8-oxoG repair after incubation of the template with nuclear extracts from liver and brain of seven week old (black bars) and 52 week old (grey bars) control (C) and hHD (Tg) mice. Data were collected from three mice, in three independent repetitions performed in parallel by two different investigators. FIG. 1D contains graphs plotting the percentage of repair of 5-hydroxycytosine (5-OHC), 3-methyladenene (3-MeA), formamidopyrimidine (FAPY) and Uracil lesions following incubation of template DNA containing the respective lesions with extracts from the brains of seven week old (black bars) and 52 week old (grey bars) control (C) and hHD (Tg) mice

FIG. 2A contains a schematic diagram of the in vitro oxidation/repair assay. FIG. 2B contains an autoradiograph of repair products obtained by incubating oxidized plasmid DNA with PC-12 cell extracts, digesting precipitated plasmid DNA with Not1 to isolate the CAG repeat tract and separating the reaction mixture on a denaturing PAGE gel. Two independent reactions were analyzed. The size of the labeled 700 bp restriction fragment is indicated. FIGS. 2C and 2D contain GeneScan traces of the distributions of CAG repeats in fibroblasts and lymphoblasts, respectively, from HD patients that were treated in culture with the indicated concentrations of hydrogen peroxide. The vertical line in each trace designates the midpoint of the CAG repeat distribution of untreated cells. FIG. 2E contains GeneScan traces of the distributions of GAA repeats in fibroblasts from HD patients treated in culture with the indicated concentrations of hydrogen peroxide. Expansion was relative to the GAA distribution of untreated cells. FIG. 2F contains photomicrographs of fluorescent images of cells treated with the indicated dose of H₂O₂ and analyzed using the Comet assay.

FIGS. 3A and 3B contain a series of GeneScan traces of CAG repeat distributions in tissues of hHD transgenic mice (wild type for OGG1) and hHD/OGG1(−/−) mice at 25 weeks of age. Each vertical dashed line designates the major size of the CAG repeat tract in corresponding young animals three weeks of age (Tail 1). The size of the repeat tract was determined by alignment with a size standard (in red). The degree of expansion is measured as the rightward shift of the midpoint with age. The size of the initial repeat (Tail 1) can be different among animals. Therefore, the size of the expansions among different tissues is determined within an individual animal of comparable age (FIGS. 3A and 3B). The results are expressed as the change in repeat size, which normalizes the data and allows for comparison of expansions among animals, independent of their starting repeat length. FIGS. 3C and 3D contain bar graphs representing the mean length change in distribution of CAG repeats in tissues from hHD mice (FIG. 3C) and hHD/OGG1(−/−) mice (FIG. 3D) at the indicated ages. Each value is expressed as the mean change and standard deviation of results from at least four mice. FIGS. 3E-3G contain representative traces of CAG repeat distributions in the tissues of hHD transgenic mice (FIG. 3E), hHD/AAG(−/−) (FIG. 3F) or hHD/Nth(−/−) (FIG. 3G) at 27 weeks of age. FIGS. 3H-3J contain bar graphs representing the quantified data from FIGS. 3E-3G, respectively. The bar graphs represent the mean length changes in distribution of CAG repeats from hHD mice (wild type for DNA repair enzymes; FIG. 3H), hHD/AAG(−/−) (FIG. 31), or hHD/Nth(−/−) (FIG. 3J) at 27-30 weeks of age. Designations and analysis are the same as in FIGS. 3C and 3D. FIGS. 3K-3M contain GeneScan traces of the distributions of repeats at the CAG locus at chromosome 9 (FIG. 3K), the CAG locus at chromosome 7 (FIG. 3L), and the polyA locus at chromosome 17 (FIG. 3M). The “n” represents the number of respective repetitive units. The size of the amplified products is indicated in base pairs at the top of the scans. The vertical dashed lines designate the major peak in the GeneScan traces.

FIG. 4A contains a schematic diagram of templates for the in vitro BER reaction. FIG. 4B contains a schematic diagram of the BER reaction. FIGS. 4C-4D contain autoradiographs of polyacrylamide gels separating reaction products of the random and CAG templates after cleavage with purified OGG1 (FIG. 4C) followed by addition of APE (FIG. 4D) to the reaction depicted in FIG. 4C. FIG. 4E, top contains schematic diagrams of templates with a 12 nt or a 6 nt gap in front of a CAG hairpin. FIG. 4E, bottom contains an autoradiograph of a polyacrylamide gel separating the hairpin templates with unpaired gaps filled in by Polβ. FIG. 4F contains autoradiographs of polyacrylamide gels separating extension products produced in the BER in vitro reaction in the absence of ligase 1 using random (left panel) or CAG (right panel) templates with purified OGG, APE, and increasing amounts of Polβ (as indicated). The positions of the 22 nt cleaved product and unligated insertion products (+1, +3, etc.) are indicated. Open circles depict major products in each lane. FIG. 4G contains autoradiographs of polyacrylamide gels separating ligated products longer than the uncleaved 160 nt starting material indicated by the arrow. The bracket depicts expansion products observed in the reaction with the CAG template. FIG. 4H is a schematic of the “Toxic Oxidation Cycle” model for somatic expansion. Oxidative lesions, such as 8-oxoG, occur frequently within CAG tracts and tend to accumulate with age. Under conditions of normal BER, OGG1/APE cleavage produces a nick, which facilitates hairpin formation and allows strand displacement during gap-filling synthesis. The lifetime of the hairpin is prolonged by MSH2/MSH3 binding, allowing ligation of extra DNA. Expansion occurs during gap-filling synthesis and ligation of the hairpin loops into the genome. Endogeneous damage arising from mitochondria respiration propagates a new cycle of oxidation to produce progressive and age-dependent mutation.

### DETAILED DESCRIPTION

This document provides methods and materials related to treating trinucleotide repeat conditions. For example, this document provides methods and materials related to using an OGG1 inhibitor to reduce progression of trinucleotide repeat expansion. The term “trinucleotide repeat condition” as used herein refers to any condition that is associated with expansion of a trinucleotide repeat sequence. A trinucleotide repeat condition can be, without limitation, fragile X syndrome, fragile XE syndrome, Friedreich ataxia, myotonic dystrophy, spinocerebellar ataxia (SCA) type 8, spinobulbar muscular atrophy (SBMA, also known as Kennedy's disease), Huntington disease (HD), dentatorubral-pallidoluysian atrophy (DRPLA), and the SCA types 1, 2, 3, 6, 7, and 12. In some cases, a trinucleotide repeat condition can be a CAG repeat condition (e.g., HD).

As described herein, an OGG1 inhibitor can be used to treat a trinucleotide repeat condition. The term “OGG1 inhibitor” as used herein refers to any agent having the ability to reduce an OGG1 polypeptide activity. Such inhibitors can include, without limitation, agents capable of inhibiting a glycolytic activity (e.g., an activity that cleaves the C1 glycoytic bond) of an OGG1 polypeptide (e.g., anti-OGG1 polypeptide antibodies), agents capable of inhibiting a lyase activity (e.g., an activity that cleaves a backbone between a ribose sugar and a 3′ phosphate) of an OGG1 polypeptide (e.g., anti-OGG1 polypeptide antibodies), agents capable of interfering with the binding of an OGG1 polypeptide to an OGG1 polypeptide substrate (e.g., anti-OGG1 polypeptide antibodies or nucleic acid containing one or more 8-oxo-G bases), agents capable of reducing transcription of nucleic acid encoding an OGG1 polypeptide (e.g., PNA oligomers targeted to the promoter region of nucleic acid encoding an OGG1 polypeptide), and agents capable of reducing expression of an OGG1 polypeptide (e.g., anti-sense oligonucleotides, siRNA molecules, or ribozymes). In addition, an OGG1 inhibitor can be identified using any of the screening methods described herein.

In some cases, an OGG1 inhibitor can be a substituted pyrrolidine. For example, an OGG1 inhibitor can be a substituted pyrrolidine as disclosed elsewhere (U.S. Pat. No. 6,369,237). In some cases, an OGG1 inhibitor can be nitric oxide (NO), peroxynitrite, or an agent that increases NO production, and can be obtained as described elsewhere (Jaiswal et al., Cancer Research, 61:6388-6393 (2001)). For example, an OGG1 inhibitor can be an agonist of NO synthase or an agent that increases the concentration of NO in cells.

In some cases, an OGG1 inhibitor can be an agent that reduces the level of mRNA that encodes an OGG1 polypeptide. For example, an OGG1 inhibitor can be an agent that reduces transcription of nucleic acid encoding an OGG1 polypeptide, or promotes degradation of mRNA encoding an OGG1 polypeptide (e.g., by RNA interference (RNAi)), or inhibits posttranscriptional processing (e.g., splicing, nuclear export) of mRNA encoding an OGG1 polypeptide. An OGG1 inhibitor can inhibit protein synthesis from OGG1 mRNA (e.g., by RNA interference), or promote the degradation of OGG1 protein, thereby reducing the level of OGG1 polypeptide in a mammal. For example, an OGG1 inhibitor can be a small interfering RNA molecule (siRNA). siRNAs can be synthesized in vitro or made from a DNA vector in vivo. In some cases, an siRNA molecule can contain a backbone modification to increase their resistance to serum nucleases and increase their half-life in the circulation. Such modification can be made as described elsewhere (Chiu et al., RNA, 9:1034-1048 (2003); and Czauderna et al., Nucleic Acids Res., 31:2705-2716 (2003)). In some cases, a small hairpin RNA (shRNA, which can be converted to an siRNA) can be used as an OGG1 inhibitor.

In general, a trinucleotide repeat condition can be treated by contacting a mammal having a trinucleotide repeat condition with an OGG1 inhibitor. Any method can be used to contact the mammal with a compound such as an OGG1 inhibitor. For example, OGG1 inhibitors can be administered orally or can be injected intramuscularly, intravenously, subcutaneously. In some cases, an OGG1 inhibitor can be injected directly into the tissues affected by the condition (e.g., nerve tissue in the spinal cord, nerves that control muscle movement in the arms and legs, skeletal muscle tissue, or brain tissue, such as striatum or cerebellum). Any method can be used to deliver an OGG1 inhibitor containing nucleic acid (e.g., a vector that expresses an siRNA molecule) to a cell. For example, standard transient transfection techniques, stable transfection techniques, or viral vectors (e.g., retroviruses, adenoviruses, herpesviruses, etc.) can be used to deliver nucleic acid to cells. In some cases, a constitutive or inducible promoter system such as those described elsewhere (Paddison et al., Methods Mol. Biol., 265, 85-100 (2004)) can be used to direct expression of a nucleic acid (e.g., an siRNA molecule or ribozyme). Nucleic acids such as siRNAs can be administered using lipid-based delivery or naked nucleic acid (e.g., DNA) injection.

An OGG1 inhibitor provided herein can be administered in combination with other agents such as those having the ability to improve a mammal's health or treat a trinucleotide repeat condition (e.g., HD). For example, an OGG1 inhibitor can be administered with an siRNA designed to reduce expression of mutant human huntingtin (htt) polypeptides. Such an siRNA can be obtained as described elsewhere (Harper et al., Proc. Natl. Acad. Sci. U.S.A., 102(16):5820-5825 (2005)). In some embodiments, multiple OGG1 inhibitors can be used.

Before administering an OGG1 inhibitor to a mammal, the mammal can be assessed to determine whether or not the mammal has a trinucleotide repeat condition. Any method can be used to determine whether or not a mammal has a trinucleotide repeat condition. For example, a mammal (e.g., a human) can be identified as having a trinucleotide repeat condition using a genetic test. For HD, a genetic test can include determining whether or not a mammal has a number of CAG repeats within the HD gene region that has been associated with HD. For example, a mammal having 28 or fewer CAG repeats can be a mammal free of HD, while a mammal having 40 or more CAG repeats can be a mammal having HD (Huntington's Disease: Hope Through Research, by NINDS, NIH Publication No. 98-49).

To determine whether or not a mammal has a trinucleotide repeat condition, a brain imaging test can be performed. For example, computed tomography (CT), magnetic resonance imaging (MRI), or positron emission tomography (PET) can be used to provide images of brain structures with little, if any, discomfort. Those mammals with HD can exhibit shrinkage of some parts of the brain such as the caudate nuclei and putamen and can exhibit enlargement of fluid-filled cavities within the brain called ventricles. These changes do not definitely indicate HD, however, because they can also occur in other disorders. In addition, a person can have early symptoms of HD and still have a normal CT scan.

Other diagnostic methods can be performed. In the example of HD, a neurologist can interview a human to obtain a medical history and rule out other conditions. A tool used by physicians to diagnose HD is to examine the human's family history. A doctor can ask about recent intellectual or emotional problems, which can be an indication of HD. In some cases, a doctor can examine a mammal's hearing, eye movements, strength, coordination, involuntary movements (chorea), sensation, reflexes, balance, movement, and mental status.

After identifying a mammal as having a trinucleotide repeat condition, the mammal can be treated with an OGG1 inhibitor. An OGG1 inhibitor can be administered to a mammal in any amount, at any frequency, and for any duration effective to achieve a desired outcome (e.g., to reduce the progression of a trinucleotide repeat expansion). In some cases, an OGG1 inhibitor can be administered to a mammal to reduce progression of symptoms of a CAG repeat condition. For example, the progression of symptoms can be reduced such that no additional progression is detected. Any method can be used to determine whether or not the progression of symptoms is reduced. For example, a person's intellectual or emotional state before and after treatment with an OGG1 inhibitor can be assessed and compared. In some cases, the number of trinucleotide repeats (e.g., CAG repeats) before and after treatment can be determined and compared. Little or no repeat expansion following the treatment can indicate that the treatment is reducing progression of the trinucleotide repeat condition. In some cases, the number of trinucleotide repeats during or following treatment can be compared to the number observed in a control population of untreated mammals having the trinucleotide repeat condition. In some cases, the degree of trinucleotide repeat (e.g., CAG repeat) expansion that occurs in one time period (e.g., 3, 6, 9, 12, or more months) during or after treatment can be determined and compared to the degree of trinucleotide repeat expansion that occurred in a time period of identical length before treatment to determine whether or not trinucleotide repeat expansion was reduced.

Brain imaging (e.g., CT, MRI, or PET) can be used to monitor the effects of a treatment. For example, brain images can be compared to those of a control population, or those taken before treatment, to evaluate the efficacy of a treatment.

Various factors can influence the efficacy of a particular treatment. For example, when a treatment is less than desired, the frequency of administration, duration of treatment, amount of an OGG1 inhibitor being administered, or route of administration can be changed. In some cases, the particular OGG1 inhibitor can be changed to a different OGG1 inhibitor.

Any method can be used to identify an OGG1 inhibitor. For example, an OGG1 inhibitor can be identified by determining whether or not a test agent reduces an OGG1 polypeptide activity, or whether or not a test agent reduces trinucleotide repeat expansion in a cell as compared to a control cell not treated with the test agent. In some cases, a potential OGG1 inhibitor can be rationally designed based on, for example, coordinates obtained from a crystal structure of an OGG1 polypeptide such as the crystal structure of native human OGG1 or human OGG1 crosslinked to 8-oxo-G DNA (Bjoras et al., J. Mol. Biol., 317(2):171-177 (2002); and Banerjee et al., Nature, 434:612-8 (2005)). Potential OGG1 inhibitors can be identified by testing individual compounds or by screening combinatorial libraries, as well as virtual combinatorial libraries as described elsewhere (Lengauer et al., Drug Discov. Today, 9(1):27-34 (2004)). When a potential OGG1 inhibitor is obtained by virtue of its ability to bind an OGG1 polypeptide, the binding interaction can be confirmed by, for example, gel shift assays. A potential inhibitor that binds an OGG1 polypeptide can be tested to determine whether or not it can reduce trinucleotide repeat expansion in a cell, in a reconstituted in vitro system (e.g., a reaction including purified components or cell extracts), or in a mammal. Positive and negative controls can be performed to assess the efficacy of a potential OGG1 inhibitor.

In some cases, siRNA molecules can be designed to serve as potential OGG1 inhibitors for testing in vitro or in vivo. Several websites as well as standard methods can be used to design siRNA molecules that target a particular polypeptide.

This document will provide additional description in the following examples, which do not limit the scope of the invention described in the claims.

### EXAMPLES

### Example 1

### Methods and Materials

Mouse Lines and Breeding

Transgenic male mice B6CBA-TgN R6/1 (Mangiarini et al., Nat. Genet., 15:197-200 (1997)) were crossed with C57BL/6J female partners that lacked one of the following glycosylases: Aag (Chong et al., Hum. Mol. Genet., 6:301- 309 (1997)), OGG1 (Klungland et al., Proc. Natl. Acad. Sci. USA, 96:13300-13305 (1999)) or Nth1 and bred to obtain homozygous knockout mice. Litters were screened for the presence of the hHD transgene and the absence of each glycosylase by PCR as described herein and elsewhere (Kovtun et al., Hum. Mol. Genet., 13:3057-3068 (2004) and Chong et al., Hum. Mol. Genet., 6:301- 309 (1997)).

Cell Cultures and Treatments

Human HD fibroblasts (Coriell Cell Repositories) were maintained in MEM medium with 20% FBS. Cells were grown until they reached 70% confluence and then treated with the indicated concentrations of H₂O₂ for 30 min. The cells were washed, the medium was replaced, and the cells were allowed to recover for three days before repeating the treatment one or two more times. After the third treatment, the cells were collected and DNA was isolated. CAG repeat sizing was performed as described below.

Comet Assay p Cells that were untreated or treated with H₂O₂ were scraped from the plates, pelleted, re-suspended in PBS (10⁴ cells/10 μl), mixed with low melting agarose (at 37° C.) and mounted on slides coated with agarose (0.5% in PBS). The cells were lysed at 4° C. for 1 hour in a buffer containing 10 mM Tris (pH 10), 2.5 M NaCl, 100 mM EDTA, and 1% triton X-100 and subjected to electrophoresis in buffer containing 300 mM NaOH and 1 mM EDTA. The slides were neutralized in 0.4 M Tris (pH 7.5) and stained with ethidium bromide (20 mg/ml). Individual cells were imaged using a LSM 510 (Zeiss) microscope with 20× objective. Fluorescence was visualized using an Ar/Kr laser at 488 nm/510 nm (excitation/emission).

In Vitro Oxidation of CAG Repeat-Containing Plasmid

A truncated version of human HD cDNA with 40 CAG repeats (Trushina et al., Proc. Natl. Acad. Sci. USA 100:12171-12176 (2003)) was inserted into the Not1 site of the pEGFP-C1 vector (Clontech). The plasmid was treated with methylene blue (10 μM in phosphate buffer, pH 7.4) and exposed to visible light for 8 min. The plasmid was then precipitated with ethanol, recovered, and used as a template in an in vitro repair reaction. Protein extract from PC-12 cells was incubated with the oxidized plasmid DNA in the repair buffer (50 mM HEPES, 5 mM MgCl₂, 2 mM DTT, 0.2 mM EDTA) with the addition of 40 mM phosphocreatine, creatine kinase, dATP, dTTP and dGTP³², dCTP³² for 1.5 hrs. Repair products were precipitated, digested with Not1 to isolate the CAG repeat tract and resolved on a denaturing PAGE gel.

Repeat Sizing

DNA was purified from mouse tissues or cultured human fibroblasts (Kovtun et al., Hum. Mol. Genet., 13:3057-3068 (2004)) and used for PCR amplification of various microsatellites. CAG repeats at the hHD locus were amplified as described previously (Kovtun and McMurray, Nat. Genet., 27:407-411 (2001); Kovtun et al., Hum. Mol. Genet., 13:3057-3068 (2004)). For amplification of GAA repeats on human chromosome 5 (accession # AC011416), the following primers were used: 5′-CCTTCTGTCTTACTTCATAG-3′ (SEQ ID NO:1) and 5′-CAGCAAAGT GTGTGTGTGGTT-3′ (SEQ ID NO:2). CAG repeats on chromosome 9 (accession #AC100550) and chromosome 7 (accession # AC122399) in the mouse genome were amplified using the primers: set 1-5′-CTCTGCACTGTGTTCAGGGAC-3′ (SEQ ID NO:3), 5′-ACTGATG CAGCCCAGGTACTG-3′ (SEQ ID NO:4) and set 2-5′-GGAAGGACCTTCATAGGCTTCT-3′ (SEQ ID NO:5), 5′-TGCCTATCTTATCC-AGCTAGGC-3′ (SEQ ID NO:6), respectively. The PolyA locus at chromosome 17 (accession # AC096777) was amplified as described (Kabbarah et al., Mol. Carcinog., 38:155-159 (2003)). One primer in each PCR amplification was labeled with FAM-6, and fluorescent products were analyzed using an ABI prism 3700 DNA analyzer instrument and the GeneMapper software v3 (Applied Biosystems).

Preparation of Nuclear Extracts and Cleavage Assay

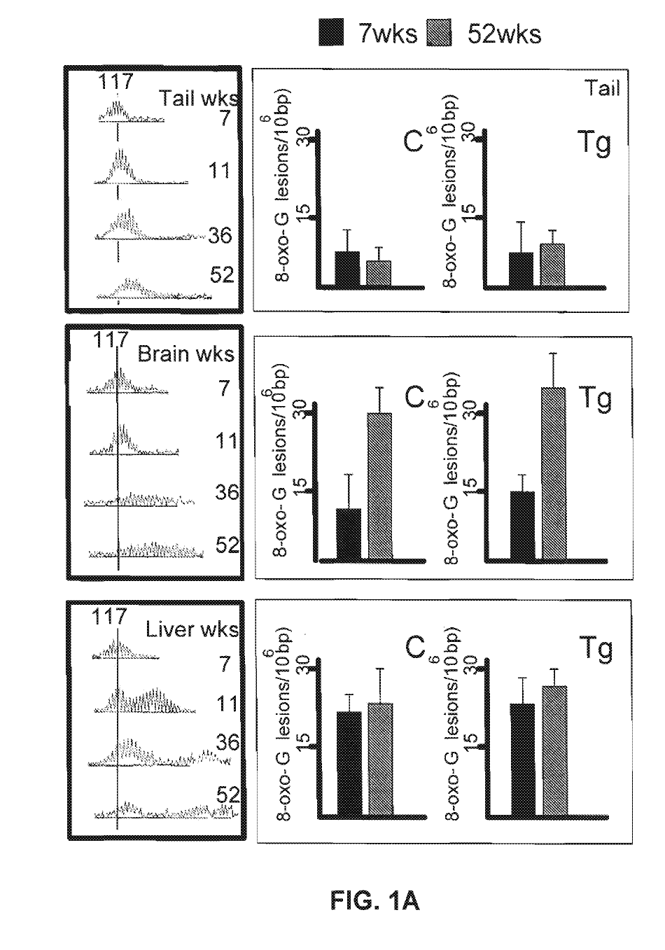

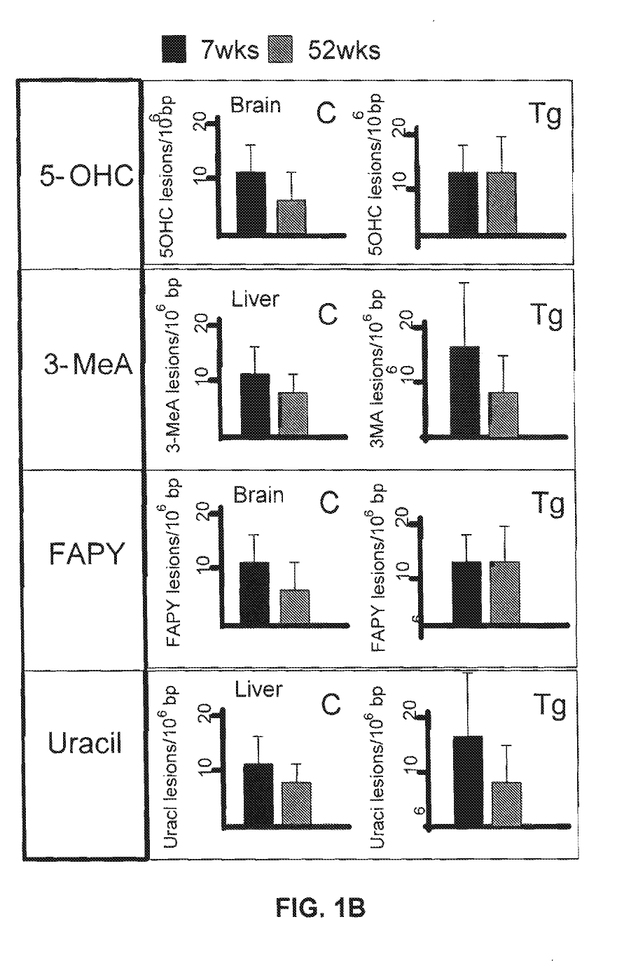

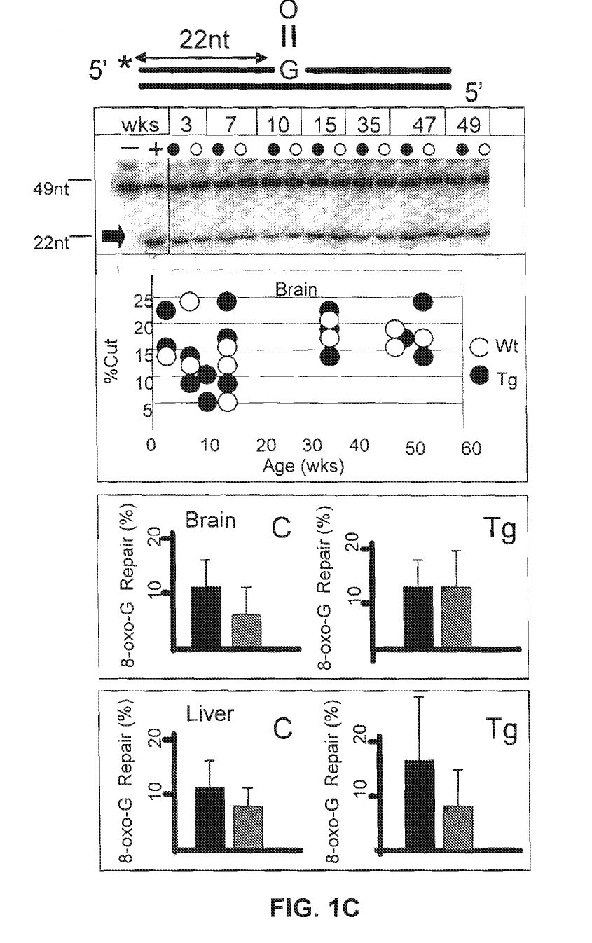

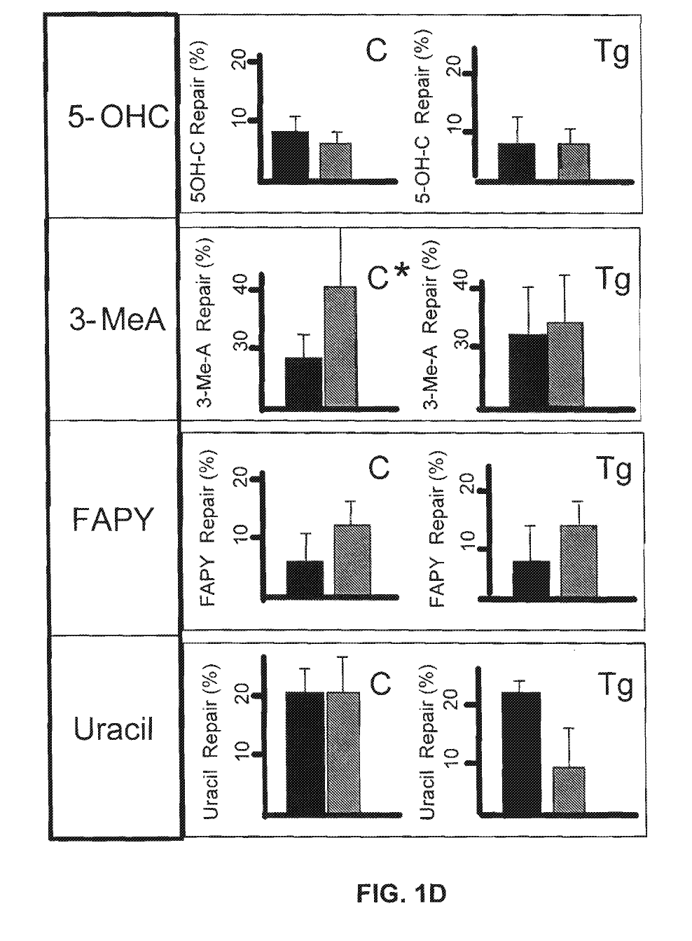

Organs were removed, quick-frozen in liquid nitrogen, and stored at 80° C. until use. Scissor-macerated tissue was passed through a 19-gauge, 1.5-inch needle. Cell suspensions were washed in hypotonic buffer containing 10 mM Hepes-KOH, pH 7.7, 0.5 mM MgCl₂, 10 mM KCl, 1 mM DTT, and 0.2 mM PMSF. The cells were lysed for 15 min. The nuclei were recovered by centrifugation at 2,000 g for 10 min. and extracted with 2 volumes of buffer containing 20 mM Hepes-KOH, pH 7.7, 0.5 mM MgCl₂, 0.42 M NaCl, 0.2 mM EDTA, 1 mM DTT, 0.2 mM PMSF, and 25% glycerol. After centrifugation at 14,000 g for 10 min., the supernatant was recovered and briefly dialyzed against buffer containing 25 mM Hepes-KOH, pH 7.7, 50 mM KCl, and 2 mM DTT. The supernatant was aliquoted and quick-frozen in liquid nitrogen. All steps were carried out at 0° C. For enzyme assays, a 49-mer oligonucleotide containing a single, centrally placed 8-oxoG residue (FIG. 1C) was ³²P-labeled at the 5′ terminus and annealed to a complementary oligonucleotide. All oligonucleotides included phosphorothioate linkages at the ultimate and penultimate 5′ and 3′ residues to reduce exonucleolytic attack. Standard reaction mixtures contained 150 fmol double-stranded oligonucleotide substrate and 2 μg nuclear extract. Oligonucleotides were recovered and resolved by denaturing 20% PAGE and visualized and quantified using a phosphorimager.

Enzyme Assays

A 49-mer oligonucleotide containing a single 8-oxoG (5′-TAGACATTGCCATTCTCGATA(8oxoG)GATCCGGTCAAACCTAGACGAATTCCG-3′; SEQ ID NO:7) was synthesized on a Perkin-Elmer/Applied Biosystems model 380B DNA synthesizer. The oligomer was deprotected with ammonia solution containing 0.25 M 2-mercaptoethanol and purified by 20% PAGE. The oligonucleotide (3 pmol) was labeled at the 5 terminus with T4 polynucleotide kinase (New England Biolabs) and [³² P]ATP (Amersham) and annealed to a complementary strand containing either an adenine (A) or a cytosine (C) residue opposite the 8-oxoG residue.

A reaction mixture (15 μL) containing 40 mM Hepes-KOH (pH 8.0), 0.1 M KCl, 0.5 mM EDTA, 0.5 mM DTT, 0.2 mg/ml BSA, and 75 fmol of ³²P-labeled 8-oxoG-containing duplex was incubated at 37° C. for one hour with purified hOGG1 (1 μg). Reactions were stopped by adding an equal volume of 95% formamide/dyes. The products were separated by gel electrophoresis and visualized by autoradiography or analyzed on a Phosphorlmager (Molecular Dynamics). Electrochemical detection of free 8-oxoG in dried ethanol supernatants of 100 μL reaction mixtures (containing phosphate buffer, pH 7, instead of Hepes, and no DTT) was performed with the Beckman HPLC Gold System using an octadecylsilane ABZ plus column (Supelco) and an electrochemical detector (model 400, EG&G, Salem, Mass.).

Preparation of Nuclear DNA and Analysis of 8-oxoG by HPLC-Electrochemical Detection (HPLC-ECD)

Male mice were sacrificed at 13-15 weeks of age. The livers were removed, quick-frozen in liquid nitrogen, and stored at 80° C. until use. Scissor-macerated liver was passed through a 19-gauge, 1.5-inch needle. Extraction of DNA and hydrolysis to nucleosides by nuclease P1 and alkaline phosphatase were performed as described previously (Zharkov et al., J. Biol. Chem., 275:28607-28617 (2000)). To reduce oxidation during DNA preparation, TEMPO (2,2,6,6-tetramethylpiperidine-N-oxyl) was added to all solutions at 100 μM immediately before use (Zharkov et al., J. Biol. Chem., 275:28607-28617 (2000)). 8-Hydroxy-2′-deoxyguanosine and 2′-deoxyguanosine were separated by HPLC and analyzed by electrochemical detection (ECD; +300 mV) and UV light (290 nm), and results were expressed as the ratio of 8-oxoG/106 bp in each DNA sample. The following conversions were used: 1 8-oxoG/105 G=4 8-oxoG/106 bp; 1 8-oxoG/106 bp=6,000 8-oxoG/diploid genome.

In Vitro Assays for Base Excision Repair

DNA oligonucleotides containing 8-oxoG were synthesized and supplied after purification by PAGE (Operon Biotechnologies Inc., Huntsville, Ala.). All other oligonucleotides were synthesized and purified by Integrated DNA Technologies Inc. (Coralville, Iowa). The 8-oxoG containing substrates were constructed by annealing the damaged strand to its template strand at a molar ratio of 1:1.5. The substrates were radiolabeled at the 5′-end of the damaged strand using [γ-³²P] ATP and Optikinase (USB Corporation, Cleveland, Ohio). Unincorporated radiolabeled material was removed with a G-25 spin column (GE Healthcare, Piscataway, N.J.). Purified human GST-tagged OGG1, APE and Pol β, at various concentrations, were employed in BER/gap-filling reactions. Purified T4 DNA ligase (60 units/μL) (New England Biolabs, Ipswitch, Mass.) was utilized in some reaction mixtures as specified. The reaction mixtures contained 50 mM Tris-HCl, pH 7.5, 50 mM KCl, 0.1 mg/mL BSA, and 0.01% NP-40. 5 mM MgCl₂ was included in APE, Pol β, and ligase reaction mixtures. 50 μM dNTPs were utilized for Pol β DNA synthesis, and 1 mM ATP was included in the ligation reaction mixtures. 10 nM oligonucleotide substrates were used in the final reaction mixture, and these substrates were pre-incubated with 30 nM OGG1 at 37° C. for 30 minutes. The final reaction mixtures (20 μL) were assembled by mixing the enzymes with the OGG1-cleaved substrates at 0° C., and then the reaction mixtures were incubated at 37° C. for 10 minutes. The reactions were terminated by addition of EDTA. The substrates and products were subjected to separation through a 15%-18% polyacrylamide denaturing gel containing 7 M urea. The products were detected by Phorsphorlmager and quantified by ImageQuant.

### Example 2

### Age-Dependent Expansion of CAG Repeats Correlates with the Level of Oxidized Bases in Vivo

To determine whether a relationship exists between age-dependent expansion of CAG repeats and oxidative damage, the level of oxidative base lesions was measured in tissues of younger (7 to 15 weeks of age) and older (15 to 52 weeks of age) hHD transgenic animals and their wild type littermates (Park et al., Proc. Natl. Acad. Sci. USA, 89:3375-3379 (1992); Helbock et al., Methods Enzymol., 300:156-166 (1999); FIG. 1A). Expansion was previously reported to appear in animals older than 15 weeks of age. Somatic tissues of hHD animals vary in the degrees of expansion (Kovtun and McMurray, Nat. Genet., 27:407-411 (2001); Mangiarini at al., Nat. Genet., 15:197-200 (1997); FIG. 1A), but tail, brain and liver were particularly informative as they reflected the broadest spectrum of change.

The level of oxidation, as judged by the level of 8-oxo-guanine (8-oxoG), was observed to be low in tail DNA and high in liver DNA. Lesions were not observed to accumulate in either of these tissues as the hHD animals aged (FIG. 1A). However, the level of the lesions in both tissues correlated with the degree of expansion. In the tail, expansion was modest at all ages tested, while in the liver, expansion began early and progressed aggressively with age (Kovtun and McMurray, Nat. Genet., 27:407-411 (2001); Kovtun et al., Hum Mol Genet., 13:3057-3068 (2004); FIG. 1A, bottom). In contrast to the tail and liver, the level of 8-oxoG in DNA from the brain accumulated 3-fold as the animals aged from 7 to 52 weeks (FIG. 1A, middle). The accumulation of 8-oxoG in the brain DNA also correlated well with the pattern of CAG expansion. Expansion was first noticeable in animals at 15-17 weeks of age and became more severe at older ages, when the degree of expansion ultimately approached that of liver. Interestingly, elevation of 8-oxoG was not limited to hHD animals. Control animals of equivalent ages accumulated the same degree of 8-oxoG in all tissues tested. These data indicated that the level of oxidation was not a specific property of transgene expression, but occurred during normal aging. To determine whether the rise in DNA damage extended to other types of base damage, the analysis was repeated for four additional DNA lesions using synthetic DNA (FIG. 1B). Briefly, duplex DNA substrates containing either a single 8-oxoG residue or a uracil site at position 14 were generated by P-end labeling the 5′-end of the 24-mer oligonucleotides (5′-GGCGGCATGACCC-[8-oxoG or uracil]-GAGGCCCATC-3′ (SEQ ID NO:8)) by T4 polynucleotide kinase (MBI Fermentas) and [γ-P] ATP (3000 Ci/mmol; Amersham). The labeled oligonucleotides were annealed to complementary strands with cytosine or guanine opposite to 8-oxoG or uracil, respectively. Similarly, a 40 bp long duplex DNA substrate containing a single 5-OHC at the indicated position (5′-AATTGCGATCTAGCTCGCCAG-[5-OHC]-AGCGACCTTATCTGATGA-3′ (SEQ ID NO:9)) was 5′-³²P-end-labeled and hybridized to a complementary strand with guanine opposite to 5-OHC. The enzyme activities were assayed in a reaction buffer containing 25 mM 3-(N-morpholino) propanesulfonic acid (MOPS), pH 7.5, 0.5 mM DTT, 0.5 mM EDTA, and 2.5% glycerol for 30 minutes at 37° C. Reaction mixtures contained 100-500 fmol substrate and 2 μg of protein extract in a total volume of 10 mL. The products of the reactions were analyzed by 20% denaturing PAGE and phosphorimaging.

N-[H3]-N methyl-N′-nitrosourea (MNU; 1.5 Ci mmol⁻¹) was used to prepare alkylated calf thymus DNA (6000 dpm mg⁻¹ DNA). 3-MeA DNA glycosylase activity was assayed in reaction buffer (70 mM MOPS, pH 7.5, 1 mM DTT, 1 mM EDTA, 5% glycerol) with 7 mg DNA substrate and 2 μg protein extract for 30 minutes at 37° C. in a total reaction volume of 50 mL. N-[H]methyl-N′-nitrosourea (18 Ci/mmol) was used to prepare poly(dG-dC) DNA containing FAPY residues (5000 dpm/mg DNA). FAPY DNA glycosylase activity was assayed in a reaction buffer (50 mM MOPS, pH 7.5, 1 mM DTT, 1 mM EDTA and 5% glycerol) with 0.4 mg FAPY substrate and 2 μg protein extract for 30 minutes at 37° C. in a total volume of 50 mL.

Following DNA precipitation of the reaction mixture, base removal was quantified as radioactivity in the supernatant by using a Liquid Scintillation Counter (Tri-Carb 2900TR, Packard).

No accumulation of 5-hyroxycytosine (5-OHC), formamidopyrimidine (FAPY), 3-methyladeneine (3-MeA), or uracil was observed in the same tissues as the animals aged (FIG. 1B). Based on these results, accumulation of 8-oxoG appeared to correlate with age-dependent expansion in the brain of hHD animals.

The rise in 8-oxoG may reflect a decrease in the capacity to repair these lesions. Alternatively, accumulation of oxidative lesions may represent an increase in endogenous oxidative damage with age. To distinguish between these two possibilities, the repair activity in brain extracts from mouse tissues of aging control and hHD animals was measured directly. Removal of oxidized bases is initiated via action of a DNA glycosylase, which excises the damaged base by cleaving the C1 glycosidic bond (Dizdaroglu, Mutat. Res., 531:109-126 (2003)). To test repair activity in mouse tissues, a DNA oligonucleotide of 49 bases was synthesized with a precisely positioned 8-oxoG in the central region of the template (FIG. 1C, top). Activity was measured by the degree of substrate cleavage after incubation with extracts of tissues from young or old animals. Successful excision generated two 5′ labeled products: a 22 nt band representing the cleaved strand and a 49 nt band representing the uncut strand (FIG. 1C, top). The products were visualized by autoradiography and quantified by phosphorimaging (Klungland et al., Proc Natl Acad Sci USA, 96:13300-13305 (1999)).

The efficiency of the repair of 8-oxoG, as judged by its excision, was not observed to differ between hHD and control animals at any age (FIG. 1C, top) in any of the tissues tested (results for brain and liver are presented in the bottom panels of FIG. 1C). Furthermore, no differences were observed between young and old animals for either control or hHD mice (FIG. 1C, bottom). To test the status of base excision overall, the same cleavage assay was used to measured the repair capacity of four additional lesions: 5-OH cytosine, 3-methyadenine, FAPY, and uracil (FIG. 1D). Consistent with the results for 8-oxoG, repair was not significantly decreased with age for any of these lesions, with the exception of uracil, for which repair appeared to be somewhat reduced in the older transgenic animals (FIG. 1D). Overall, these data indicated that the accumulation of oxidative lesions in DNA observed in vivo (FIG. 1A) was not due to an inability of the tissues to repair the lesions. Rather, the results reflected an increase in endogenous damage during normal aging.

### Example 3

### Oxidative Base Damage Leads to CAG Expansion in Vitro

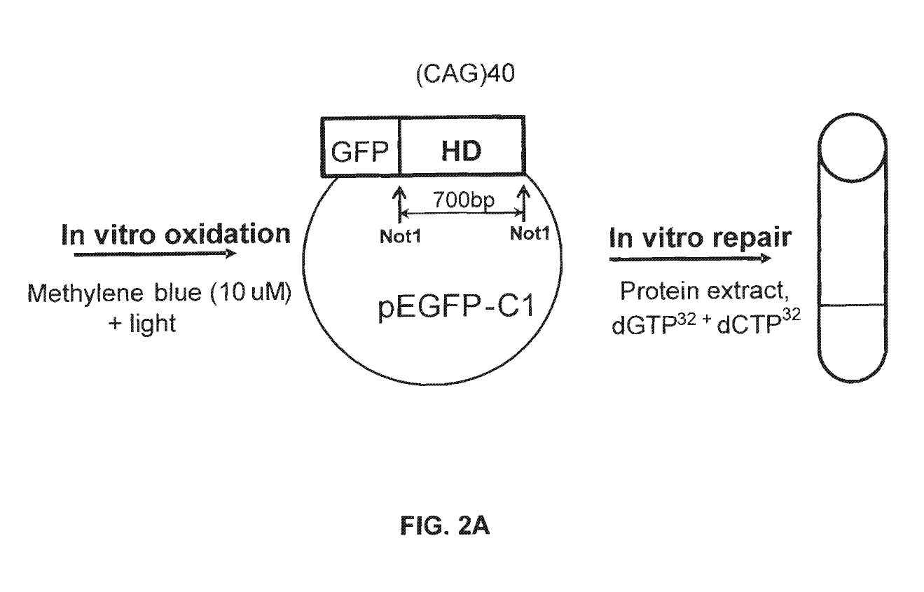

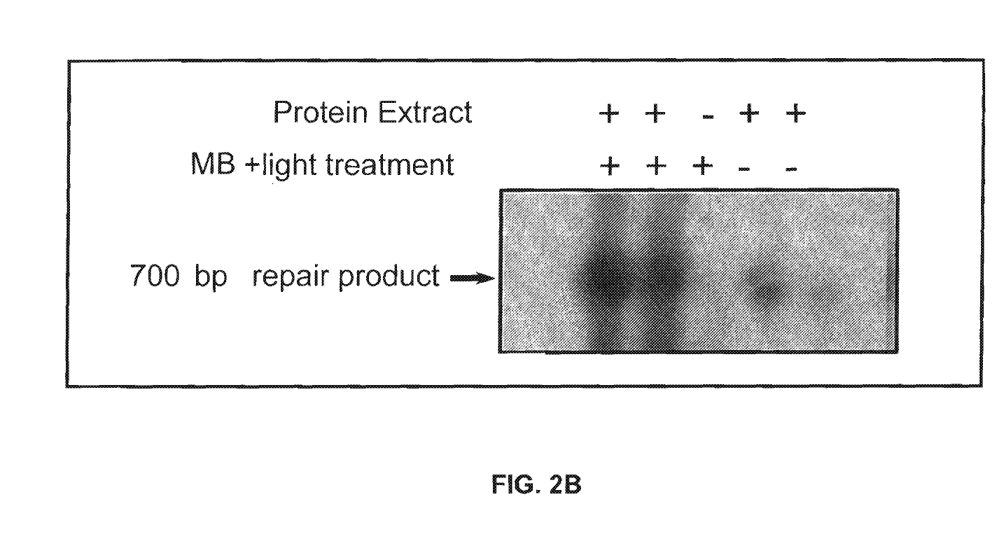

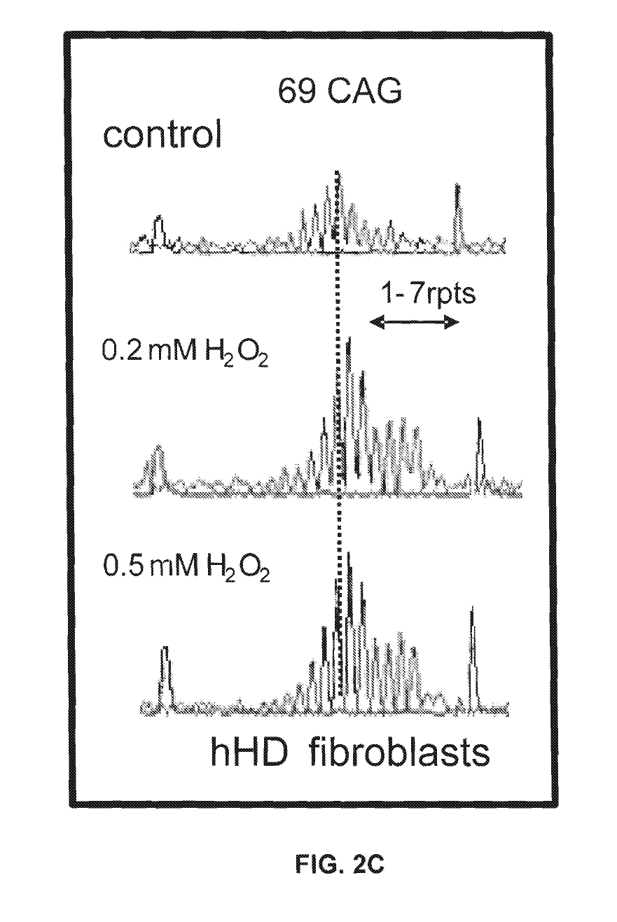

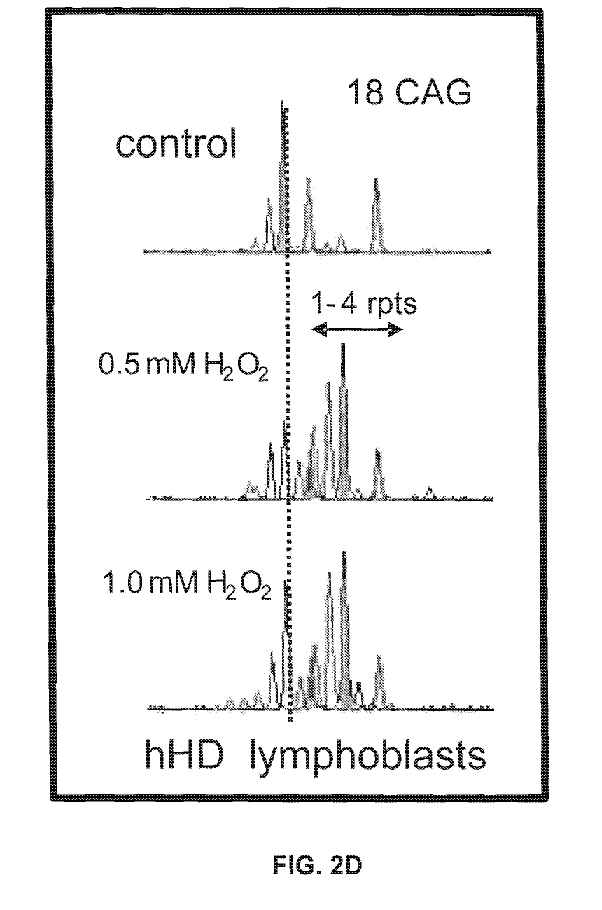

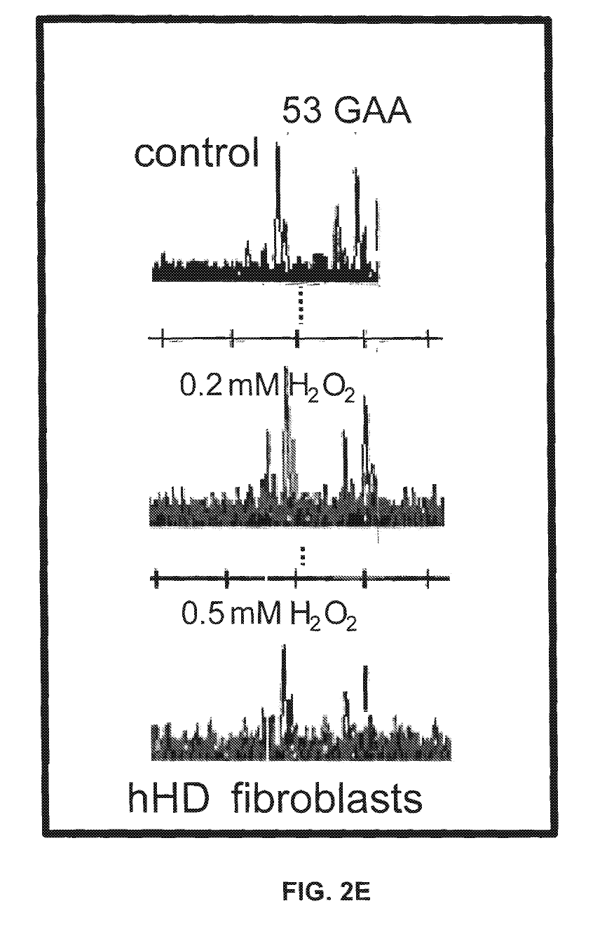

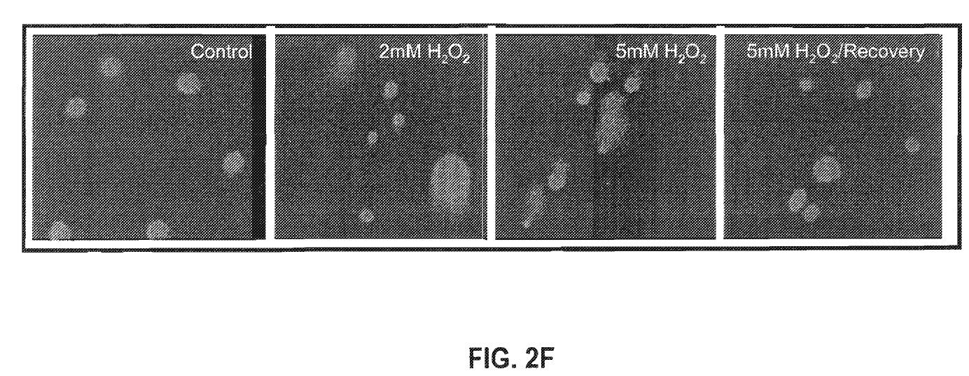

Following the observation that oxidative base damage correlated with somatic expansion in vivo (FIG. 1), it was determined whether base oxidation could directly lead to expansion. If expansion is a response to base damage, then expansion should arise if the CAG repeat tract is exposed to oxidizing agents. To test this hypothesis, fibroblasts from HD patients were challenged with sub-lethal doses of hydrogen peroxide (H₂O₂), and expansion of the CAG repeats was monitored (FIG. 2). Peroxide was chosen as the oxidizing agent since a major fraction of endogenous DNA damage arises during mitochondrial respiration when the superoxide anion radical (O2•) is converted into hydrogen peroxide (Zharkov et al., J. Biol. Chem., 275:28607-28617 (2000)).

It was first established whether the CAG repeat sequences in the hHD locus were suitable targets for oxidation and base excision repair (FIGS. 2A and 2B). An intact plasmid containing a truncated hHD gene with 40 CAG repeats was incubated with methylene blue (MB) and light in the presence of cell extracts and radiolabeled dGTP and dCTP. The following was performed to assess in vitro oxidation of CAG repeat-containing plasmid. Plasmid DNA that contained a truncated version of human HD cDNA with 40 CAG repeats (cDNA was inserted into a Not1 site of a pEGFP-C1 vector (Clontech); Trushina et al., Proc. Natl. Acad. Sci. USA, 100:12171 (2003)) was treated with methylene blue (10 μM in phosphate buffer, pH 7.4) and exposed to visible light for 8 minutes. Ethanol precipitated and recovered plasmid then was used as a template for an in vitro repair reaction. Protein extract from PC-12 cells was incubated with the oxidized plasmid DNA in the repair buffer (50 mM HEPES, 5 mM MgCl₂, 2 mM DTT, 0.2 mM EDTA) with the addition of 40 mM phosphocreatine, creatine kinase, dATP, dTTP, and dGTP³², dCTP³² for 1.5 hours. Repair products were precipitated, digested with Not1 to isolate a CAG repeat tract, and resolved on denaturing PAGE gel (FIG. 2B). Under these conditions, the excised CAG tract was strongly labeled (FIG. 2B). No incorporation was observed in the absence of either MB/light or cell extracts. These results indicated that the CAG tracts at the HD locus could be oxidized and repaired. It was then determined whether peroxide treatment could directly lead to CAG expansion at the hHD locus in human HD fibroblasts in response to oxidative damage. Indeed, treatment of the cells with low doses of H₂O₂ uniformly led to expansion of CAG at intermediate or disease-length alleles (FIGS. 2C and 2D). The results did not depend on cell type. Peroxide-induced expansions were observed in multiple human fibroblast and lymphoblast cell lines obtained from HD patients (FIGS. 2C and 2D). These data demonstrated that CAG expansion could directly arise in response to oxidative damage in vitro.

To determine whether expansion of CAG repeats at the HD locus was a unique outcome, or whether oxidation caused general genome instability in treated cells, other repetitive sites were examined. No expansions in other repetitive sites were observed. For example, GAA/CTT repeats have been reported to be the longest arrays in the genome of humans (Freudenreich et al., Science, 270:853-856 (1998)). The stability of the longest stretch of GAA repeats in the human genome, based on a search of NCBI genebank, was examined in the same cells subjected to hydrogen peroxide treatment. Despite the fact that every third base of this sequence was C or G, expansion was not detected at this locus in peroxide-treated cells (FIG. 2E). Expansion in human HD fibroblasts treated with peroxide appeared to depend on the sequence of the repeat in a manner consistent with the disease. Based on the comet assay, peroxide treatment caused a dose-dependent increase in single strand breaks in cultured cells (FIG. 2F). The selected doses did not affect cell viability, and repair of single strand breaks in peroxide treated cells was essentially complete within two hours post-treatment. These results indicated that CAG expansion occurred in cells subjected to oxidative stress under conditions of normal repair of single strand breaks. Only the CAG repeats at the HD locus expanded in vitro under conditions of oxidative damage, while other sites tested were repaired faithfully.

### Example 4

### OGG1 is Required for Age-Dependent Expansion in Vivo

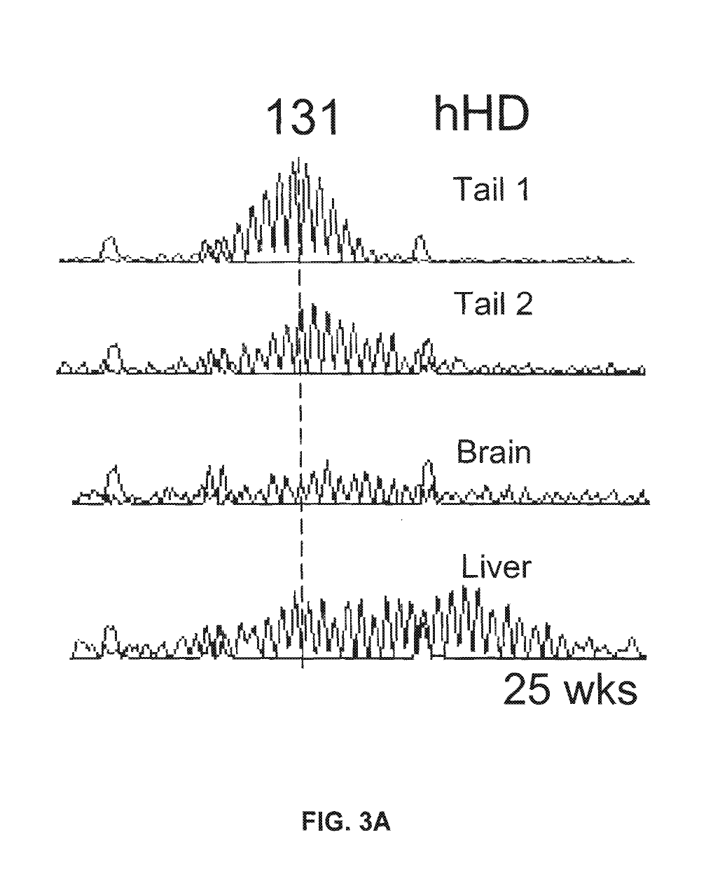

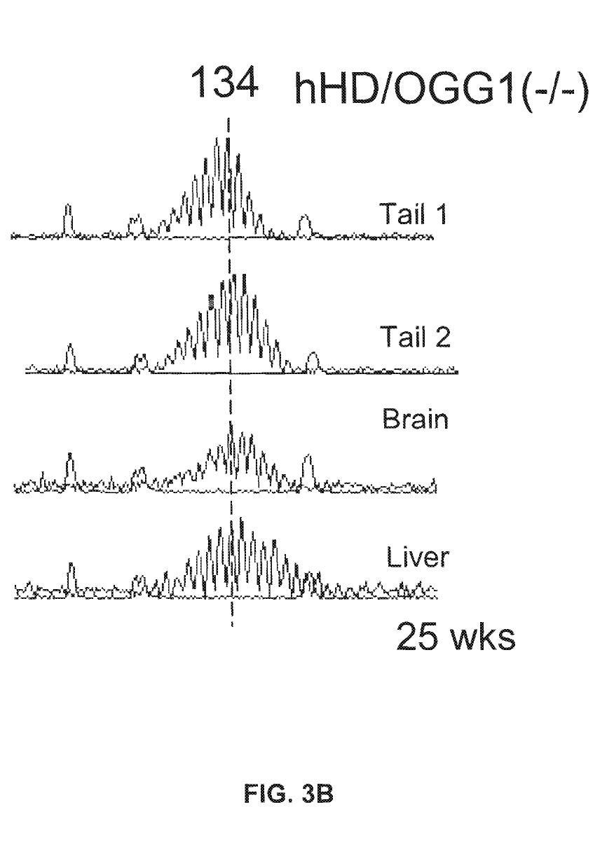

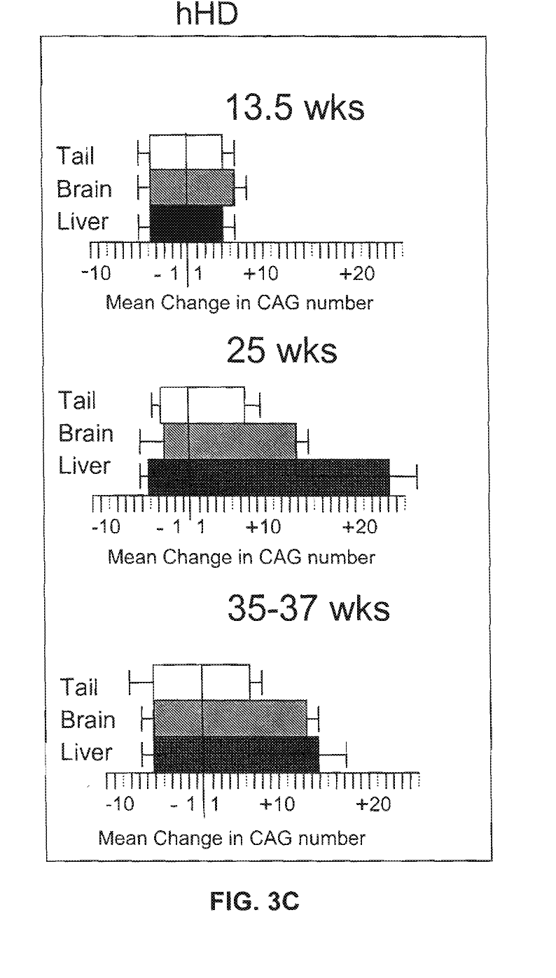

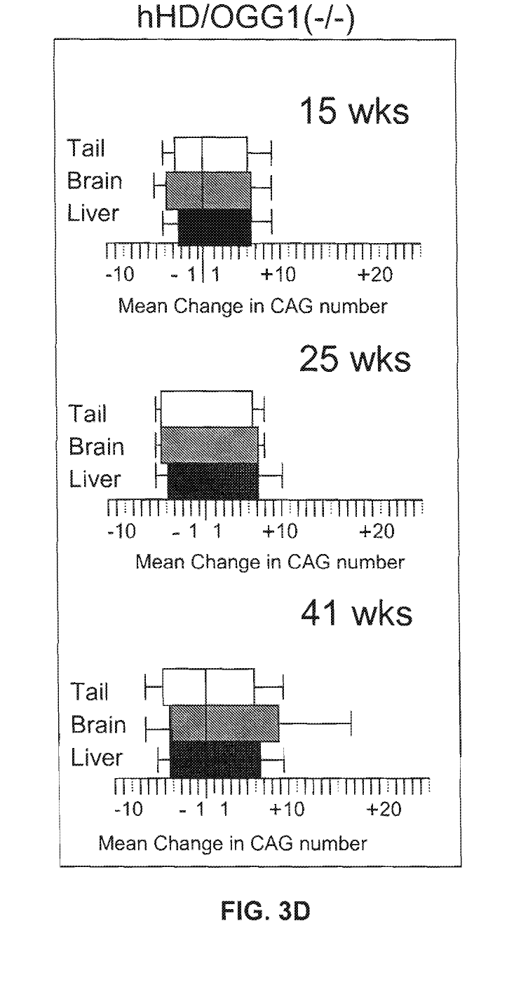

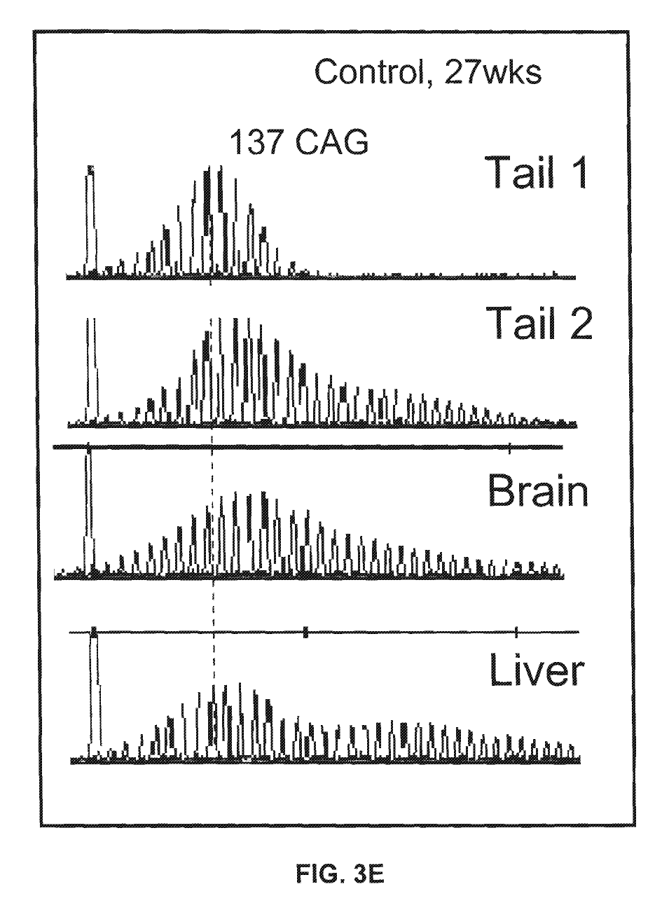

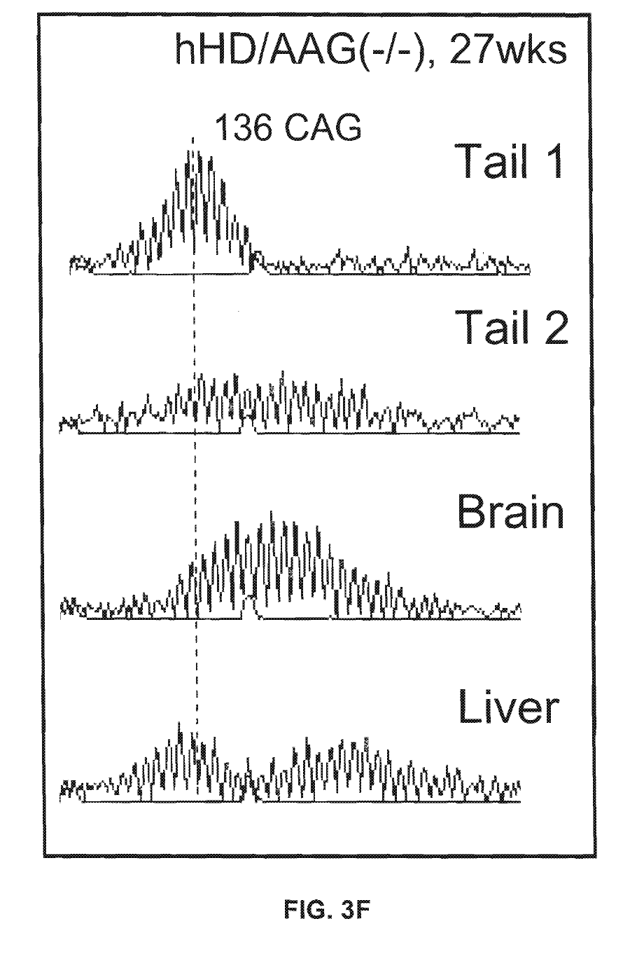

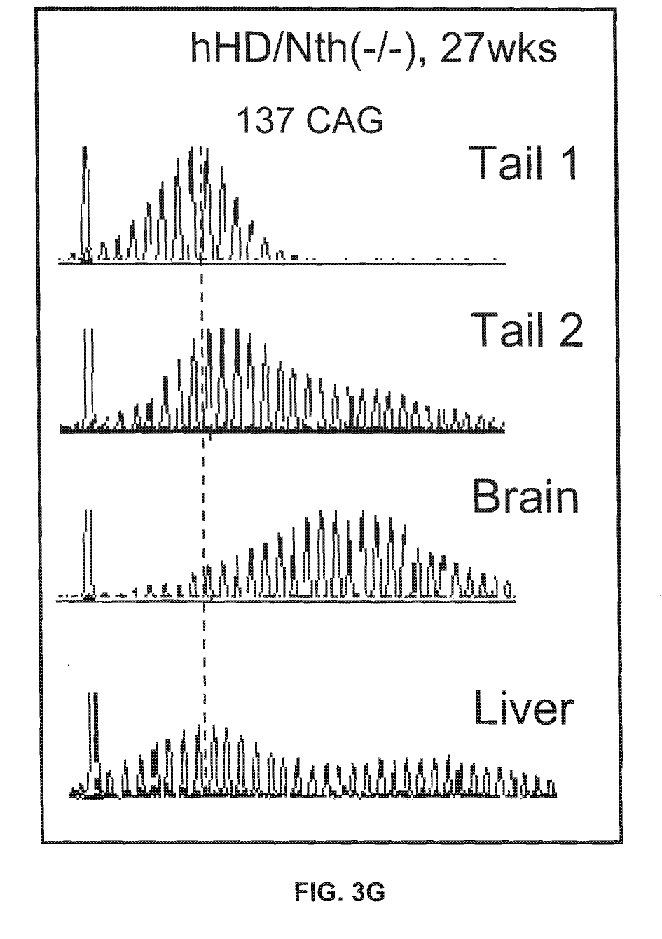

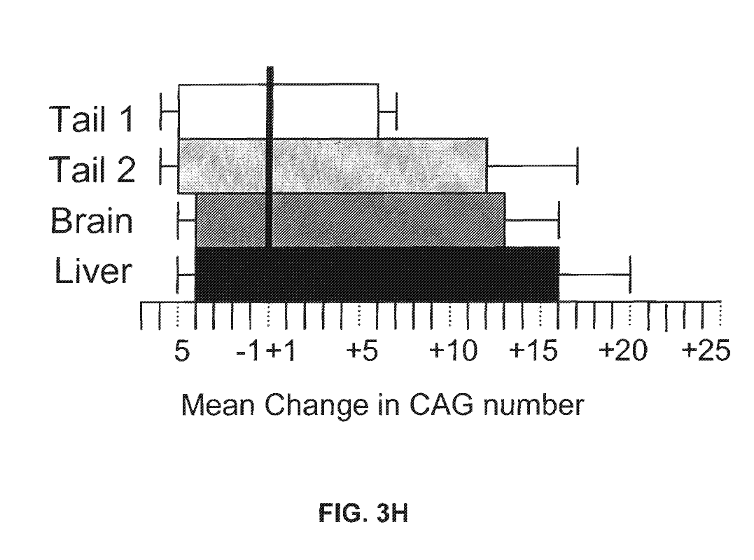

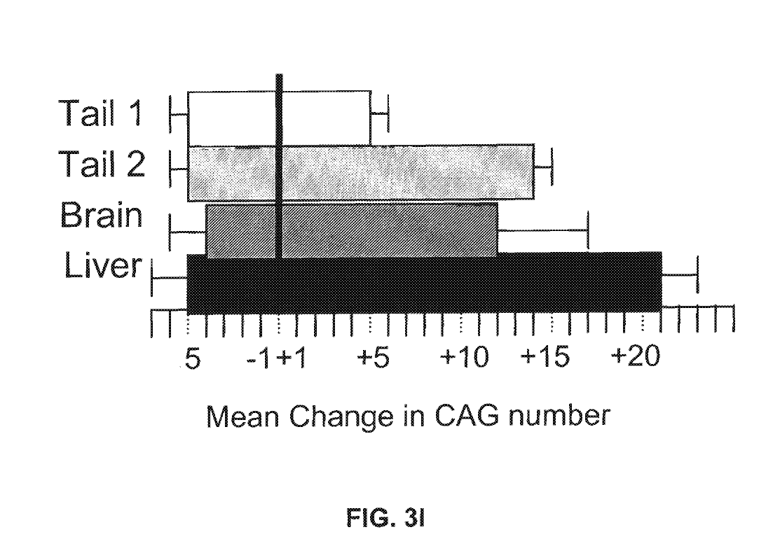

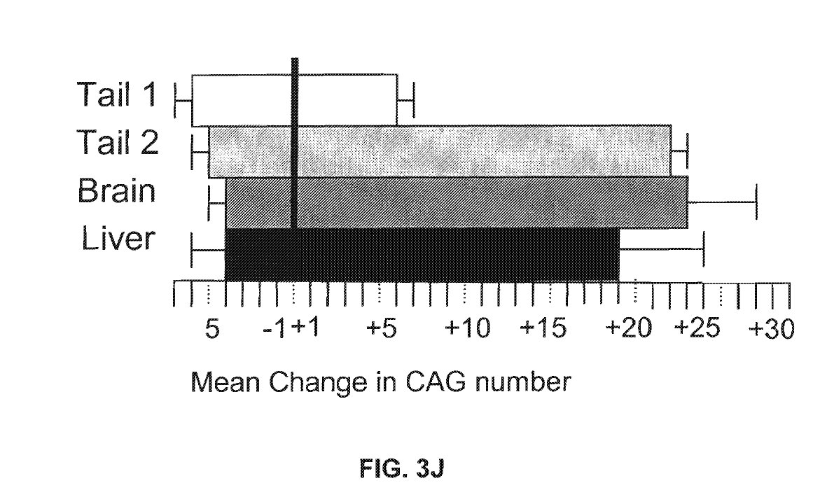

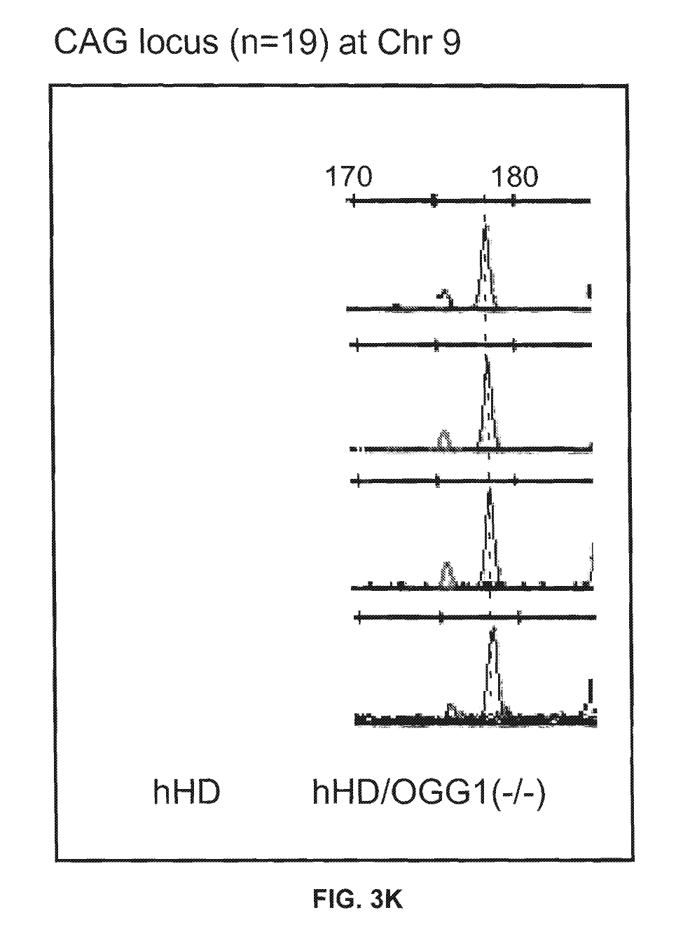

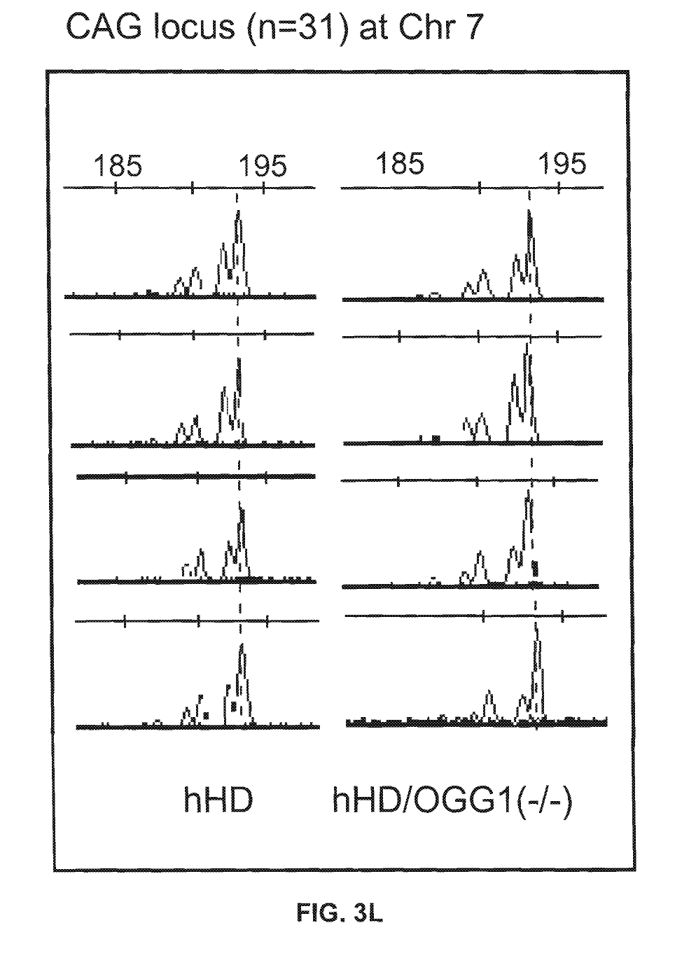

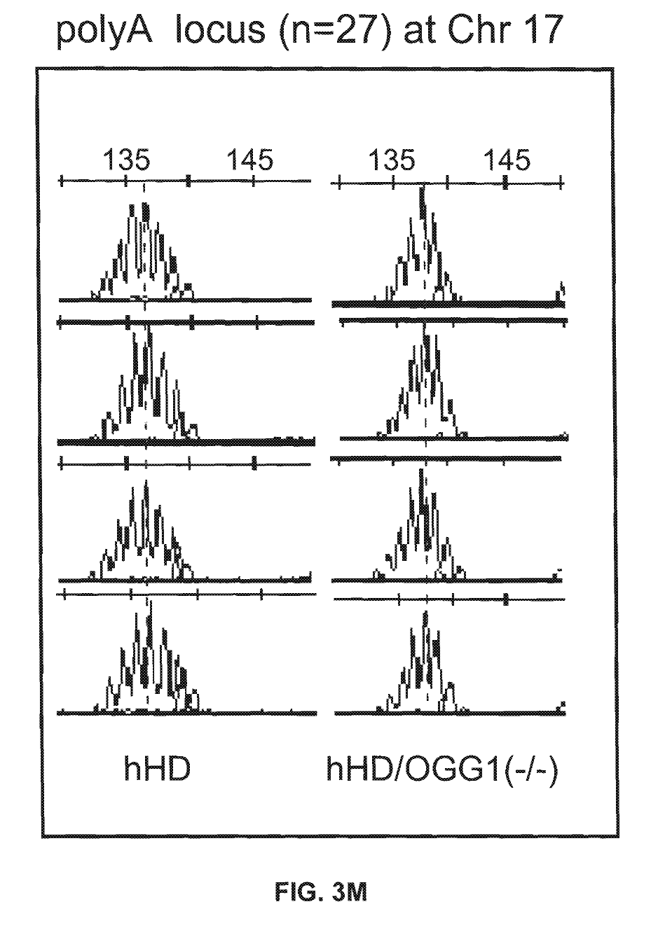

In vivo, oxidized bases are repaired predominantly via the base excision repair (BER) pathway (Dizdaroglu, Mutat. Res., 531:109-126 (2003)). This is consistent with the significant level of single stranded breaks observed in HD fibroblasts after peroxide treatment. It was determined whether expansion arises in the process of BER in vivo. If removal of oxidized bases is a crucial step in the process of expansion, then loss of the relevant glycosylase(s) could reduce age-dependent expansion. Since 8-oxoG lesions were observed to accumulate with age in mouse tissues in vivo (FIG. 1), it was determined whether OGG1, a major enzyme that recognizes and removes 8-oxoG opposite C (Klungland et al., Proc Natl Acad Sci USA, 96:13300-13305 (1999); Auffret van der Kemp et al., Proc. Natl. Acad. Sci. USA, 93:5197-5202 (1996); Mariappan et al., J. Mol. Biol., 285:2035-2052 (1999); Morland et al. Nucleic Acids Res., 30:4926-4936 (2002)), plays a role in somatic expansion. hHD mice were crossed with mice lacking OGG1, and the expansion profile at CAG repeats was measured in tissues of these mice at different ages. Surprisingly, loss of this single glycosylase significantly suppressed or delayed age-dependent expansion in vivo (FIGS. 3A-3D). At comparable ages, expansions were absent or suppressed in hHD/OGG (−/−) animals (FIG. 3B) relative to their littermate controls (FIG. 3A), despite the fact that all other glycosylases were present and might be used to repair the oxidative lesions. Although loss of OGG1 significantly inhibited expansion (FIGS. 3C and 3D), the effect was not absolute. Expansion was prevented in roughly 70% of hHD/OGG(−/−) animals. In the remaining 30% of the animals, tissue-specific expansion could be observed with age in the brain or in the liver of hHD/OGG(−/−) animals, although to a lesser extent than in the hHD littermates.

The incomplete influence of OGG1 on expansion may be due to individual differences in the level of oxidative damage or compensation of the OGG1 function by other glycosylases. Nevertheless, the significant, measurable effect on expansion in vivo due to loss of the single glycosylase, OGG1, was unexpected (FIG. 3). Most glycosylases have preferred but overlapping substrate specificity (Bjelland and Seeberg, Mutat. Res., 531:37-80 (2003); Dizdaroglu, Mutat Res., 531:109-126 (2003); Morland et al., Nucleic Acids Res., 30:4926-4936 (2002)). To further test the specificity of OGG1 in the expansion process, two additional mouse lines were created by crossing hHD mice with mice either lacking alkyl adenine glycosylase (AAG) or Nth1 (homologue of Escherichia coli endonuclease III; FIGS. 3E-3J). These two glycosylases are used in BER pathways, but they have distinct substrate preferences (Bjelland and Seeberg, Mutat. Res., 531:37-80 (2003); Dizdaroglu, Mutat Res., 531:109-126 (2003); Fortini et al., Mutat. Res., 531:127-139 (2003); Dizdaroglu et al., Biochemistry, 38:243-246 (1999); O'Brien and Ellenberger, J Biol Chem., 279:9750-9757 (2003)). Nth1 prefers to excise the lesion thymine glycol (Fortini et al., Mutat. Res., 531:127-139 (2003); O'Brien and Ellenberger, J Biol Chem., 279:9750-9757 (2003)), and cleavage of oxidized bases occurs by the same enzymatic mechanism as cleavage by OGG1. AAG excises a variety of alkylated bases and has the highest affinity for 3-methyl adenine (Dizdaroglu et al., Biochemistry, 38:243-246 (1999)). The two mouse lines created by crossing hHD mice with mice lacking either AAG or Nth1 were used to determine whether expansion depended on other glycosylases or on other types of lesions. hHD mice lacking AAG were found to expand their CAG repeat tract within the human transgene at every age tested and in every tissue tested (FIGS. 3F and 3I). These results indicated that removal of alkylated bases, which could be present in at least one nucleotide of every CAG repeat, did not contribute to age-dependent somatic expansion of long CAG repeats in the hHD locus. Similar results were obtained for hHD/Nth(−/−) mice (FIGS. 3G and 3J). No significant differences were observed in hHD/AGG(−/−) and hHD/Nth1(−/−) mice relative to their hHD littermates controls (FIG. 3). These data indicated that loss of a single glycosylase, OGG1, was a dominant factor contributing to age-dependent expansion in vivo, despite the fact that other DNA glycosylases were present and the tissues were competent for repair.

### Example 5

### The Mutation Profile of Expansion in hHD Animals is Consistent with Human Disease

Because OGG1 appeared to be a causative factor in expansion mutation, it was determined whether expansion in hHD and hHD/OGG1(−/−) mice displayed a mutation profile consistent with human disease. Four properties characterize the expansion mutation. First, expansion mutation occurs primarily at repeats capable of forming secondary structure (Kovtun et al., Biochem. Cell. Biol., 279:325-336 (2001), Chauhan et al., J Biomol. Struct. Dyn., 20:253-263 (2002)). Repeats capable of forming secondary structure increase the frequency of expansion in yeast from 5-1000-fold, while the mutation frequency of random sequence DNA or repeats with no ability to form secondary structure is distinguishable from background (Spiro et al., Mol. Cell, 4:1079-1085 (1999)). Second, expansion in human disease has a threshold CAG length, below which there is little probability of observing an expansion. Third, CAG expansion is typically observed at tract lengths above 36 repeats in humans, who have long life spans (Marenstein et al., J. Biol. Chem., 278:9005 (2003); Kremer et al., Am. J. Hum. Genet., 57:343-350 (1995)), while the threshold is around 100 in mice, which have short lifetimes. Finally, expansion is largely restricted to the disease locus and is not accompanied by general microsatellite instability at other repetitive sites. These properties could be recapitulated in vitro in peroxide treated cells. Experiments were also performed in vivo to determine whether age-dependent expansion in hHD mice was (i) length dependent, (ii) sequence dependent, and (iii) observed at other repetitive sequences.

Expansion did not occur at short CAG repeats, since it was not observed in the endogenous mouse HD gene (6 units). Expansion of unrelated alleles containing long CAG tracts was examined (FIGS. 3K-3M). The longest uninterrupted stretches of CAG repeats were identified in a mouse genome by searching the NCBI genebank. One of these stretches contains 19 CAG repeats (located on chromosome 9). A second site contains a CAG repeat of 31 (on chromosome 7). CAG repeats at both loci were found to be stable in both hHD and hHD/OGG1(−/−) mice. These results indicated that expansion in vivo was length-dependent and was restricted to repeats above the threshold, the long CAG tract (135 rpt) within the hHD transgene (FIGS. 3K-3M). Consistent with the role of secondary structure formation, no age-dependent expansion was observed at sequences lacking this capability. Results for the polyA microsatellite are presented in FIG. 3M. Similar results were observed for other microsatellites tested. These results indicated that expansion in hHD mice was also sequence-dependent. Overall, the mutation profile of expansion in hHD animals appeared to replicate length and sequence characteristics of the mutation observed in human disease: a dominant single site CAG expansion at the disease-length hHD locus as the animals aged.

### Example 6

### OGG1 Cleavage of CAG Repeat DNA can Initiate Strand Displacement and Expansion in Vitro in the Presence of Purified BER Machinery

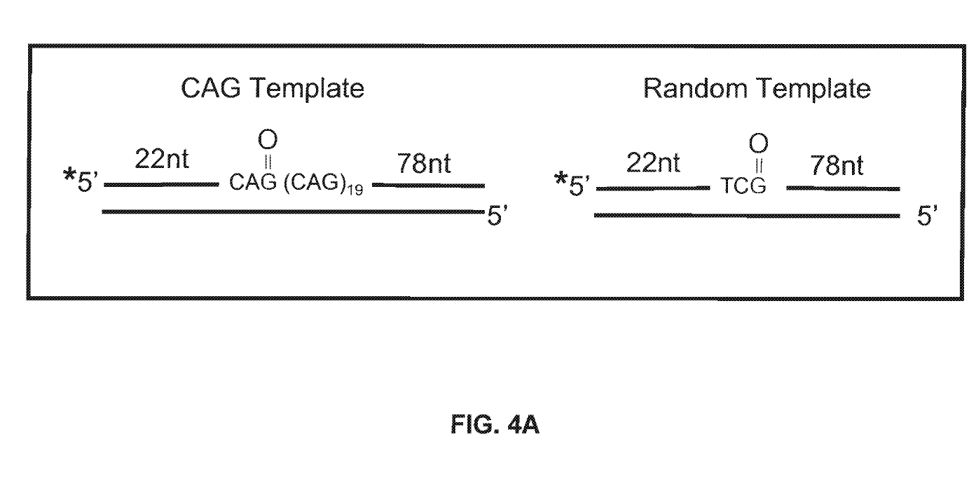

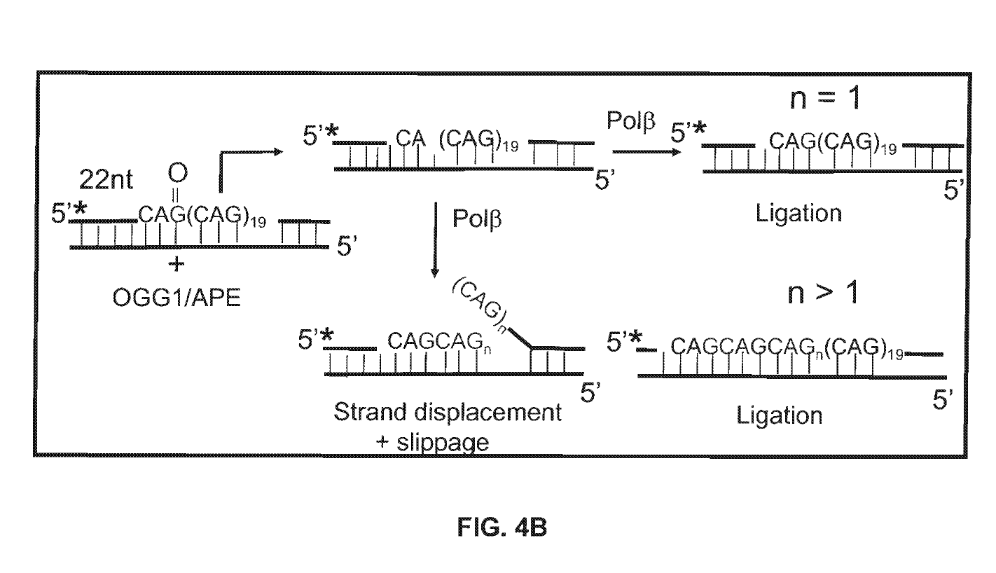

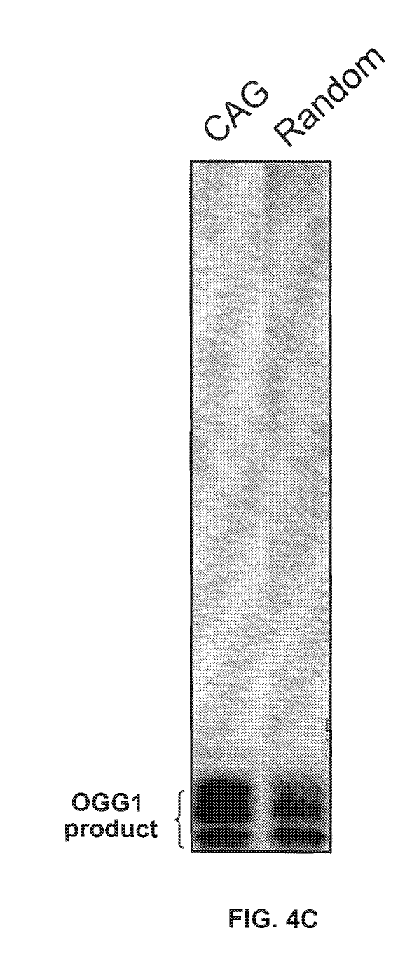

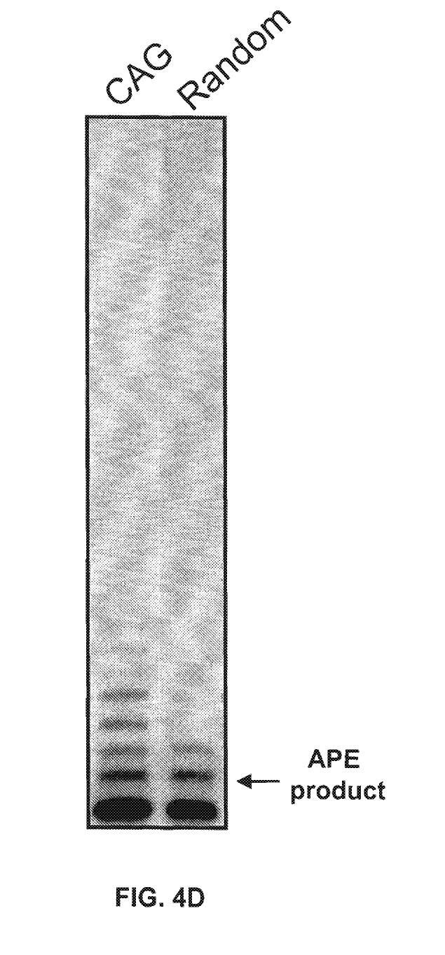

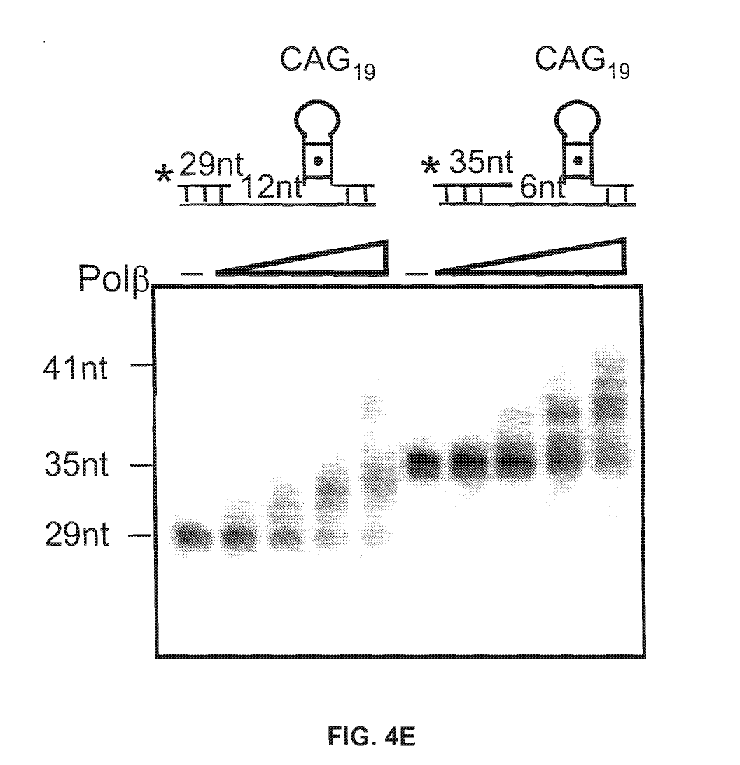

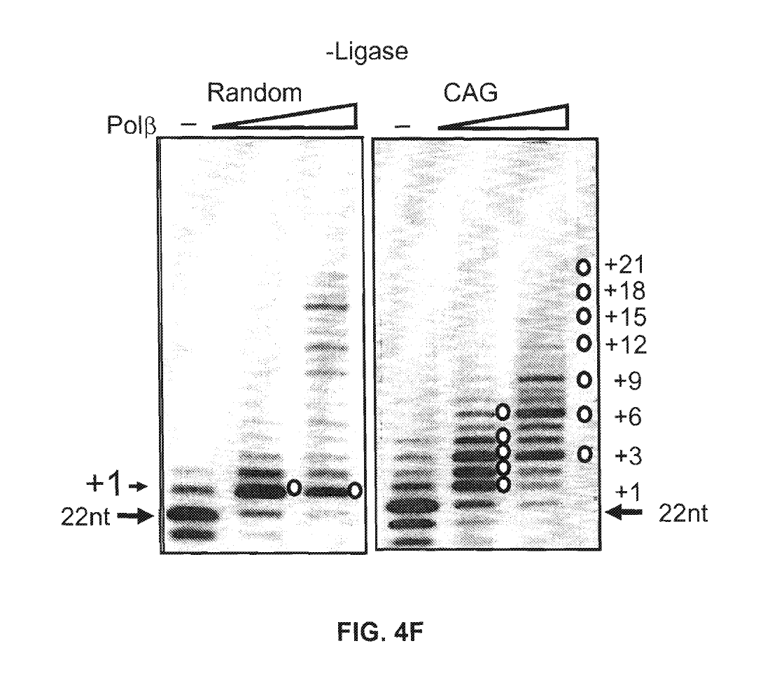

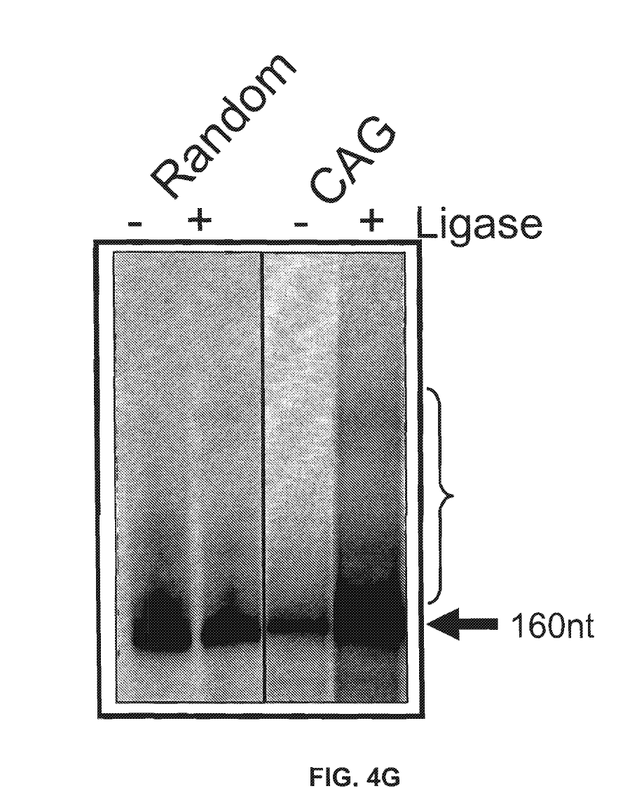

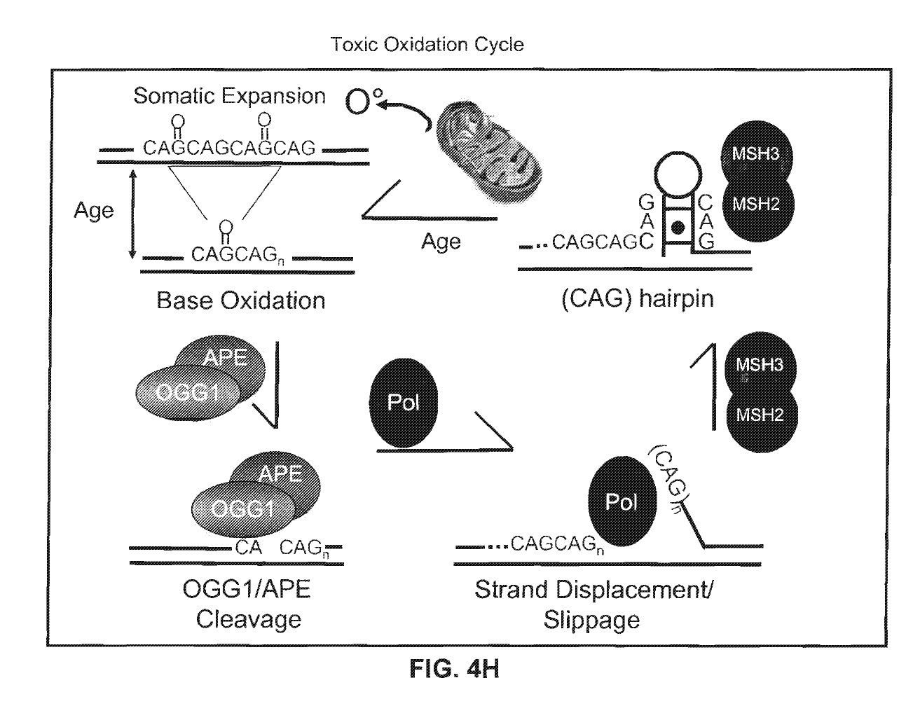

The in vivo data indicated that structure-capable CAG repeats expanded in the process of normal base excision and that OGG1 was required. Based on these results, it was of interest to determine whether expansion could be generated in vitro using purified OGG1 and purified components of the BER machinery. While polymerases are known to generate expansion on CAG templates by primer extension on open templates or on synthetic mimics of an abasic site, an assay was needed to test BER beginning at the initial step of removal of lesions within CAG duplex DNA by a glycosylase. Templates were designed and an in vitro assay was created to recapitulate the entire BER process. Two DNA templates were synthesized in which a single 8-oxoG base was positioned within a 160 bp duplex (FIG. 4A). In the CAG template, the base lesion was flanked by 19 CAG repeats (CAG template). In the control template, the base lesion was flanked by a random DNA sequence of roughly equal CG and AT content (random template). These templates were used to determine whether CAG expansion arises in vitro after OGG1 cleavage, and whether the mutation depends on the CAG repeat sequence.

Polymerase β(Polβ), which often cooperates with OGG1 to carry out BER, was used to reconstitute the BER reaction in vitro. Polβ was chosen for its propensity to repair gaps by single nucleotide additions. Excision by OGG1 followed by APE cleavage leaves a single nucleotide as a template for the gap-filling reaction. If CAG sequences are not required for expansion, then Polβ gap filling synthesis on CAG and random templates would be expected to yield primarily single nucleotide extension products (FIG. 4B). On the other hand, if CAG sequences are required for expansion, then some strand displacement would be expected to occur after OGG1/APE cleavage and Polβ synthesis. Under these conditions, gap-filling reactions by Polβ would yield extension products of more than a single nucleotide on CAG templates relative to random templates, and expansion should be observed.

It was first determined whether each component of the BER machinery functioned properly on the in vitro templates. Each template was 5′ end labeled with ³²P. After cleavage by OGG1, each template should produce two fragments observable by autoradiography: the 22 nt cleavage product and any residual 160 nt uncut strand. Purified OGG1 and APE1 were able to cleave both the random template and the CAG template, producing end-labeled fragments of the expected size (FIGS. 4C and 4D). OGG1 cleavage was efficient on both CAG and random templates as no residual 160 nt band was observed. Successful APE cleavage should remove a nucleotide from the OGG1 cleavage product, and an n−1 band (relative to the OGG1 cleavage product) was indeed observed for both the CAG and random templates. Furthermore, Polβ was unable to carry out strand extension of the 22 nt band unless APE was included in the BER reaction. As a final control, the ability of Polβ to fill gaps larger than 1-2 nt on CAG repeat DNA was tested. CAG templates were synthesized with gaps of 6, 12, and 21 nt in front of a CAG hairpin. Polβ was observed to efficiently fill unpaired gaps of 6 and 12 nt on CAG hairpin templates (FIG. 4E). Gaps of 21 nt were filled less efficiently.

Once it was determined that all of the purified BER components functioned properly on CAG repeat DNA, it was determined whether OGG1 cleavage of 8-oxoG could facilitate expansion. When OGG1 was added together with a random template, the major product observed at all ratios of Polβ to DNA template tested was a +1 extension (FIG. 4F). At the highest ratio (1:1), some strand displacement occurred on the random template, but the +1 addition product still dominated. In contrast, at an equivalent concentration, Polβ produced longer additions on CAG templates (FIG. 4F). At low Polβ to CAG template ratios, the +1 extension product was observed, but the +2 and +3 products matched its intensity. Minor but significant bands were also observed as +4 to +6 additions. At the highest Polβ/CAG template ratio, there was little +1 band detected. Instead, a series of products spaced in a triplet pattern were observed, consistent with favored addition of a CAG unit (FIG. 4F). To determine whether strand displacement of CAG extension products could be ligated and lead to expansion, the BER assay analysis was repeated in the presence of ligase (FIG. 4G). Products generated by Polβ extension of the CAG template produced a set of labeled products of CAG bands with sizes larger than the 160 nt uncut strand (FIG. 4G). In contrast, few ligation products could be detected on random templates under the same conditions (FIG. 4G). These data indicated that CAG repeats can facilitate strand displacement during Polβ gap filling synthesis, and the displaced strands can be successfully ligated to produce CAG expansion products.

### Example 7

### Assays to Determine the Efficacy of OGG1 Inhibitors

The activity of an OGG1 inhibitor is assessed in vivo using Huntington's mice. The mice are separated into two groups. Mice in one group are treated with an OGG1 inhibitor, while mice in the other group are treated with a placebo. Tissues are isolated from mice in both groups at various time points. PCR amplification is used to monitor the number of CAG repeats in the tissues over time, as the mice age. A decreased level of expansion of CAG repeats in the animals treated with the OGG1 inhibitor compared to the placebo-treated controls indicates that the OGG1 inhibitor is effective, whereas a comparable or increased level of expansion in OGG1 inhibitor-treated animals compared to placebo-treated animals indicates that the OGG1 inhibitor is not effective. Additional controls include treated and untreated wild-type, OGG1(−/−), and HD/OGG(−/−) animals. The same in vivo assay is also used to assess prevention of the expansion of CAG repeats in animals exposed to environmental and toxic oxidizing agents (e.g., paraquat) and treated with an OGG1 inhibitor.

The activity of an OGG1 inhibitor is also assessed using an in vitro DNA repair assay. Oligonucleotide templates containing precisely positioned 8-oxoG bases are synthesized. The oligonucleotide templates are end-labeled and incubated with cell extracts from wild-type, HD, OGG1(−/−), and HD/OGG(−/−) animals that are mock-treated or treated with an OGG1 inhibitor. Repair is measured by detecting cleavage products corresponding to the position of the 8-oxoG. The degree of cleavage is a measure of the extent to which the lesion is repaired. A decreased level of cleavage of templates that are incubated with tissue extracts from animals containing OGG1 that are treated with the OGG1 inhibitor, as compared to the level of cleavage of templates incubated with tissue extracts from mock-treated animals containing OGG1, indicates that the OGG1 inhibitor is effective. A comparable or increased level of cleavage of templates incubated with tissue extracts from animals containing OGG1 that are treated with the OGG1 inhibitor, as compared to the level of cleavage of templates incubated with tissue extracts from mock-treated animals containing OGG1, indicates that the OGG1 inhibitor is not effective.

The activity of an OGG1 inhibitor is also assessed in vitro using fibroblasts from HD patients. The fibroblasts are treated with oxidizing agents. In addition, the fibroblasts are mock-treated or treated with an OGG1 inhibitor. PCR amplification is used to monitor the degree of expansion of CAG repeats in the mock-treated and the OGG1 inhibitor-treated cells. A decreased level of expansion of CAG repeats in the OGG1 inhibitor-treated cells compared to the mock-treated cells indicates that the OGG1 inhibitor is effective, whereas a comparable or increased level of expansion in OGG1 inhibitor-treated cells compared to mock-treated cells indicates that the OGG1 inhibitor is not effective.

The activity of an OGG1 inhibitor is also assessed in vitro using a luciferase assay. A plasmid expressing the luciferase gene is transfected into cells in culture. The cells are treated with one or more oxidizing agents, and the consequent reduction in luciferase expression is measured. The cells are also mock-treated or treated with an OGG1 inhibitor. The expression of luciferase is again monitored. Recovery of luciferase expression indicates repair of oxidative damage. Thus, the degree of recovery of luciferase expression is a measure of the level of OGG1 activity in the cells. A decreased level of luciferase expression in cells treated with the OGG1 inhibitor compared to mock-treated cells indicates that the OGG1 inhibitor is effective. A level of luciferase expression in OGG1 inhibitor-treated cells that is comparable to or greater than the level of luciferase expression in mock-treated cells, and greater than the level of luciferase expression immediately following oxidative damage, indicates that the OGG1 inhibitor is not effective.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing progression of a CAG repeat condition in a mammal, said method comprising administering an inhibitor of an OGG1 polypeptide activity to said mammal under conditions wherein progression of symptoms of said CAG repeat condition is reduced as compared to progression of symptoms in a control mammal having said CAG repeat condition and not having been administered said inhibitor.

2. The method of claim 1, wherein said CAG repeat condition is Huntington's disease.

3. The method of claim 1, wherein said mammal is a human.

4. The method of claim 1, wherein said inhibitor is a substituted pyrrolidine.

5. The method of claim 1, wherein said inhibitor reduces the level of an OGG1 polypeptide in said mammal.

6. The method of claim 1, wherein said inhibitor reduces the level of mRNA that encodes an OGG1 polypeptide in said mammal.

7. The method of claim 5, wherein said inhibitor induces RNA interference.

8. The method of claim 1, wherein said inhibitor is a nucleic acid comprising an 8-oxo-guanine base.

9. The method of claim 1, wherein progression of said CAG repeat condition is stopped in said mammal.

10. The method of claim 1, wherein said method comprises diagnosing said mammal as having said CAG repeat condition.

11. A method for identifying a potential agent for reducing progression of a CAG repeat condition, said method comprising:
(a) determining whether or not a test agent inhibits an OGG1 polypeptide activity, and
(b) determining whether or not said test agent reduces CAG repeat expansion in a cell compared to a control cell not treated with said test agent, wherein the presence of a reduced CAG repeat expansion indicates that said test agent is said potential agent.

12. The method of claim 11, wherein said CAG repeat condition is Huntington's disease.

13. The method of claim 11, wherein said test agent is a substituted pyrrolidine.

14. The method of claim 11, wherein said test agent comprises nucleic acid.

15. The method of claim 11, wherein said step (a) comprises determining whether or not said test agent reduces the level of an OGG1 polypeptide in cells.

16. The method of claim 11, wherein said step (a) comprises determining whether or not said test agent reduces the level of mRNA that encodes an OGG1 polypeptide in cells.

17. The method of claim 11, wherein said test agent is a nucleic acid comprising an 8-oxo-guanine base.

18. The method of claim 11, wherein said cell is in vitro.

19. The method of claim 18, wherein said cell is isolated from a mouse having said CAG repeat condition.

20. The method of claim 18, wherein said cell is a mouse embryonic fibroblast cell.

21. The method of claim 11, wherein said cell is in a mammal.

22. The method of claim 21, wherein said mammal is a mouse.

23. The method of claim 21, wherein said mouse is a mhtt mouse.

24. The method of claim 23, wherein said mhtt mouse is an R6/1 mouse.
